(19) United States
(12) Patent Application Publication
(10) Pub. No.: US 2026/0031214 A1
(43) Pub. Date: Jan. 29, 2026

(54) SYSTEMS FOR GENERATING PERSONALIZED DIETARY SUPPLEMENTS

(71) Applicant: GULP INDUSTRIES, INC., Salt Lake City, UT (US)

(72) Inventor: Bradley Burr MCLAWS, Salt Lake City, UT (US)

(21) Appl. No.: 18/996,618

(22) PCT Filed: Jul. 17, 2023
(86) PCT No.: PCT/US2023/070349

§ 371 Date: Jan. 17, 2025

Related U.S. Application Data
(60) Provisional application No. 63/390,145, filed on Jul. 18, 2022.

(51) Int. Cl. G16H 20/60; A61K 31/047; A61K 31/05; A61K 31/07; A61K 31/10; A61K 31/12; A61K 31/122; A61K 31/164; A61K 31/197; A61K 31/198; A61K 31/202; A61K 31/205; A61K 31/216; A61K 31/355; A61K 31/375; A61K 31/4045; A61K 31/405; A61K 31/4188; A61K 31/4375; A61K 31/4415; A61K 31/455; A61K 31/4745; A61K 31/51; A61K 31/519; A61K 31/525; A61K 31/566; A61K 31/575; A61K 31/593; A61K 31/7008; A61K 31/714; A61K 33/06; A61K 33/22; A61K 33/24; A61K 33/26; A61K 33/30; A61K 33/32; A61K 35/00; A61K 35/745; A61K 35/747; A61K 35/748; A61K 36/05; A61K 36/064; A61K 36/07; A61K 36/074; A61K 36/15; A61K 36/16; A61K 36/185; A61K 36/21; A61K 36/23; A61K 36/258; A61K 36/31; A61K 36/41; A61K 36/42; A61K 36/45; A61K 36/48; A61K 36/481; A61K 36/53; A61K 36/534; A61K 36/537; A61K 36/575; A61K 36/63; A61K 36/67; A61K 36/68; A61K 36/71; A61K 36/73; A61K 36/736; A61K 36/81; A61K 36/82; A61K 36/84; A61K 36/87; A61K 36/88; A61K 36/889; A61K 36/9068; G16H 10/60

(52) U.S. Cl. CPC G16H 20/60; G16H 10/60; A61K 31/047; A61K 31/05; A61K 31/07; A61K 31/10; A61K 31/12; A61K 31/122; A61K 31/164; A61K 31/197; A61K 31/198; A61K 31/202; A61K 31/205; A61K 31/216; A61K 31/355; A61K 31/375; A61K 31/4045; A61K 31/405; A61K 31/4188; A61K 31/4375; A61K 31/4415; A61K 31/455; A61K 31/4745; A61K 31/51; A61K 31/519; A61K 31/525; A61K 31/566; A61K 31/575; A61K 31/593; A61K 31/7008; A61K 31/714; A61K 33/06; A61K 33/22; A61K 33/24; A61K 33/26; A61K 33/30; A61K 33/32; A61K 2035/115; A61K 35/745; A61K 35/747; A61K 35/748; A61K 36/05; A61K 36/064; A61K 36/07; A61K 36/074; A61K 36/15; A61K 36/16; A61K 36/185; A61K 36/21; A61K 36/23; A61K 36/258; A61K 36/31; A61K 36/41; A61K 36/42; A61K 36/45; A61K 36/48; A61K 36/481; A61K 36/53; A61K 36/534; A61K 36/537; A61K 36/575; A61K 36/5777; A61K 36/63; A61K 36/67; A61K 36/68; A61K 36/71; A61K 36/73; A61K 36/736; A61K 36/81; A61K 36/82; A61K 36/84; A61K 36/87; A61K 36/88; A61K 36/889; A61K 36/9068

(57) ABSTRACT

Techniques for personalizing dietary supplements for use across multiple user profiles and for personalizing a set of dietary supplements for a specific user are disclosed. Information detailing a health profile element is received. Based on that health profile element, certain therapeutic targets are identified. A data structure is accessed, where this structure maps various therapeutic conditions to dietary supplements designed to alleviate those conditions. Formulas of dietary supplements are optionally multi-purposefully over-formulated. A limited number of differing types of prepackaged units of formulas of dietary supplements is identified. A set of at least two prepackaged units is selected. This set constitutes a divided daily dosage for the user. The set also includes dietary supplements designed to alleviate the therapeutic targets of the user. Dynamically personalized labels for the set are generated.

### BACKGROUND

A dietary supplement is an ingredient or combination of ingredients a person can add to his/her diet to promote health or reduce the risk of health problems. One example of a dietary supplement is a multivitamin. Vitamins, minerals, and other nutrients are critical to maintaining a person's health and normal biochemical functions. Dietary supplements are colloquially referred to as simply “supplements” or “vitamins,” even when some do not technically include vitamins. A “multivitamin” is a type of dietary supplement that combines in a single dosage form a plurality of dietary supplement ingredients.

Currently, there are different types of multivitamins formulated for different broad categories of customers. For instance, there are often multivitamin products marketed as being targeted to men, to women, to children, or to older individuals. The blends or mixtures of vitamins and minerals that are included in these respective products are usually varied slightly based on the target group for whom the product is designated. As an example, the amount of iron may be higher in a multivitamin targeted to women to help alleviate potential anemia issues. The amount of vitamin D and magnesium may be higher in a multivitamin product targeted to men to help with blood pressure issues.

The multivitamin industry has conventionally focused on providing a “one size fits most” approach for the supplement ingredients they provide in a multivitamin product. For instance, the general class of multivitamins for “men” is a very large class. In this sense, the industry purports to provide customized multivitamins, but the granularity of that customization is extremely course and does not reach a level that truly represents “personalized” supplements.

Although other approaches aiming to increase the level of customization of supplement products have been proposed, such approaches have thus far failed to provide a workable solution. That is, prior approaches have failed to provide (1) sufficient customization to rise to the level of truly personalized supplements, while also (2) being economically feasible and practically implementable in the marketplace.

Accordingly, there is an ongoing need for systems and methods that improve the art of customized supplements. The subject matter claimed herein is not limited to embodiments that solve any disadvantages or that operate only in environments such as those described above. Rather, this background is only provided to illustrate one exemplary technology area where some embodiments described herein may be practiced.

### BRIEF SUMMARY

Embodiments disclosed herein relate to systems, devices, and methods for generating sets of “personalized” dietary supplement ingredients for use across multiple profiles and for personalizing a particular set of dietary supplement ingredients for a specific user. As used herein, such “personalized” supplements are those that have a level of customization sufficient to be substantially unique to the individual. This is used in contrast to conventional “customized” supplements which are only customized with respect to very broad categories of people (e.g., men's formulas, women's formulas, old age formulas). The embodiments described herein thus make use of customization, but the customization is sufficiently granular to generate “personalized” supplements.

Embodiments disclosed herein utilize one or more of the following three principles, which will be described in more detail below, to enable effective personalization of supplements: (1) combinatorial daily dosing, (2) intelligent formulation, and (3) dynamic personalization.

Some embodiments receive information detailing a health profile element of a user. Based on the health profile element, the embodiments then identify one or more therapeutic conditions associated with the user. The embodiments also access a data structure that maps each therapeutic condition to a corresponding set of one or more dietary supplement ingredients designed to address that therapeutic condition. In some cases, a “therapeutic target” is a subset of possible “therapeutic conditions” that may be addressed. These therapeutic targets have a relationship with raw profile data (e.g., they can represent the profile data points from the raw data, which is actionable). The identified therapeutic targets (identified from the set of therapeutic conditions) can thus be mapped to a corresponding set of dietary supplement ingredients designed to address that therapeutic target.

Different formulas of dietary supplement ingredients are included in different sets of prepackaged units that are intelligently formulated. For example, the prepackaged units can be multi-purposefully over-formulated such that the formulas contained therein can address multiple therapeutic conditions. In some optional scenarios, a machine learning engine can be involved in generating and/or updating the data structure, and the machine learning engine can optionally use user feedback to generate and/or update the data structure. Machine learning techniques can be applied, for example, to analyzing user surveys and/or other sources of user health information, the generation of user profiles based on received health information, the identification of therapeutic targets for a user, the mapping of supplement ingredients to user profiles based on their identified therapeutic targets, and the intelligent generation of formulations for the prepackaged units assigned to the users.

The embodiments can identify a number of differing types of prepackaged units of dietary supplement ingredients. The dietary supplement ingredients of the prepackaged units are selected from the data structure. The embodiments select, from among the limited number of differing types of prepackaged units, a set of at least two prepackaged units. The selection process is based on the health profile element and on the data structure. The set of at least two prepackaged units together constitutes a divided daily dosage for the user. The divided daily dosage is formulated to provide the identified, recommended set of supplement ingredients for addressing the user's therapeutic targets.

The embodiments can also generate Supplement Facts labels for the set of at least two prepackaged units. Here, the Supplement Facts labels are designed to identify correlations between the dietary supplement ingredients included in the set of at least two prepackaged units and the one or more therapeutic targets of the user. Therefore, despite the dietary supplement ingredients (that are included in the set of at least two prepackaged units) being usable to alleviate multiple different therapeutic conditions, the Supplement Facts labels are designed to emphasize the correlations that are relevant to the specific therapeutic targets of the user. As explained in more detail below, these correlations can be illustrated on the generated Supplement Facts label as a listed set of proprietary blends. The blend names (e.g., immunity blend, athletic performance blend, cognition blend, etc.) represent the therapeutic targets of the user or generalized categories of the therapeutic targets of the user. This personalized information can be displayed on the prepackaged units themselves, and can additionally or alternatively be displayed on a website (e.g., as part of the user profile information accessible by the user), on packaging materials (e.g., an insert), a flyer, and the like.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the invention may be realized and obtained by means of the instruments and combinations particularly pointed out in the appended claims. Features of the present invention will become more fully apparent from the following description and appended claims, or may be learned by the practice of the invention as set forth hereinafter.

### BRIEF DESCRIPTION OF THE DRAWINGS

In order to describe the manner in which the above-recited and other advantages and features can be obtained, a more particular description of the subject matter briefly described above will be rendered by reference to specific embodiments which are illustrated in the appended drawings. Understanding that these drawings depict only typical embodiments and are not therefore to be considered to be limiting in scope, embodiments will be described and explained with additional specificity and detail through the use of the accompanying drawings in which:

FIGS. 1A and 1B illustrate an example architecture that can be used to generate personalized multivitamins.

FIGS. 2A and 2B illustrates an example data structure showing a mapping between therapeutic conditions and supplement ingredients.

FIG. 3 illustrates an example process flow for customizing multivitamins.

FIG. 4 illustrates a principle focused on combinatorial daily dosing.

FIG. 5 illustrates how many different profiles can be serviced by the disclosed principles.

FIGS. 6A and 6B illustrate a principle focused on intelligent formulation, which can involve multi-purpose over-formulation.

FIGS. 7A and 7B illustrate a principle focused on dynamic personalization.

FIG. 8 illustrates a flowchart of an example method for personalizing supplements.

FIG. 9 illustrates an example computer system capable of performing any of the disclosed operations.

### DETAILED DESCRIPTION

### Conceptual Overview

A multivitamin can be generic to the population (i.e., a one-formula-fits-all formula) or it could be formulated to address a group with common health needs. For instance, the general class of multivitamins for “men” or multivitamins for “women” can broadly customize the multivitamins to those groups. However, these are very large groups, and the level of customization is thus very low. Ideally, supplements would include a formula that is specifically suited to the specific heath needs of a single individual based on a sophisticated and detailed health profile. A multivitamin formula ideally includes a large number of clinically proven supplement ingredients targeted to support an individual's varied spectrum of health problems, concerns, goals, history, and/or risk factors. This objective is widely appreciated as the “Holy Grail” of the supplement and multivitamin industry. However, because of logistical impracticalities and economic infeasibility (including shelf space and inventory limitations), the granularity of customization of multivitamins has been highly restricted.

The historic problem of creating a truly personalized multivitamin product (i.e., a multivitamin formula precisely created to suit to the health needs of a single individual) involves the issue of creating one defined unit (stock keeping unit (SKU), which represents a defined bottle, package, etc.) for one “class” (or profile/type/group) of individuals. Practical considerations of inventory cost, inventory space, and manufacturing capability limit the number of product types (i.e., different types of SKUs), and thus the number of different classes that can be served with a customized multivitamin formula. To serve just one random individual among billions of possible customers with an individualized formula capable of treating a nearly infinite unique combination of health targets would require the manufacture and storage of a nearly infinite number of SKUs.

A multivitamin product could be precisely and uniquely formulated for a small group or even an individual, but this would require the ability to predict and manufacture in advance (or in real time) billions of different formulas to fit each possible individual. This is practically impossible. To illustrate, the number of different formulas needed is a function of how many variables are used to describe the person. For example, if you considered just three broad health variables, each with only two possibilities: age (young/old), gender (male/female), and diabetes status (yes/no), you would need eight formulas to target the eight possible groups/classes defined by these three variables (two ages groups times two gender groups times two diabetes status groups). Again, these eight example formulas are not very personalized. The actual size of any one of those eight groups may be larger than a billion people, for example. This example is provided to illustrate the math associated with variables that define an individual's health profile.

Truly personalizing a multivitamin formula requires a description of hundreds of health variables (e.g., diet, genetic mutations, weight/height, exercise behaviors, sun exposure, blood sugar levels, various family histories of heart, mood and brain health, immunity strength, medicine taken, concussion received, allergies, sleep patterns, sexual health needs, inflammation levels, cholesterol levels, homocysteine levels, environmental and biological stressors, energy levels, toxin and germ exposure, past infections or respiratory conditions, headaches, joint pains, and/or skin dryness), each having two or more possibilities. If you used hundreds of variables to define or describe an individual's health profile it would result in a nearly infinite number of groups/classes of people. Although this level of granularity could address variants in multivitamin customers down to or near the level of a single individual, it is impossible to implement. This level of therapeutic precision has been the goal for supplement manufacturers. However, conventional approaches have been unable to develop a logistically practical and economically feasible approach for accomplishing such personalization of multivitamins.

True personalization (as that term is defined herein) would require billions of formulas to be made in advance, with prepackaged units of those formulas available on shelves awaiting that unique person to call on, order, or buy that unique product. A manufacturer would have to plan for, formulate, manufacture, and store these formulas. This is simply not possible. Billions of SKUs would need to be produced. Most of those products would never be purchased by the theoretical individual to which they correspond. Huge numbers of bottles/units would expire before being purchased and would be dumped in landfills. Moreover, it is impossible to provide sufficient retail shelf space (even in a virtual environment) for these billions of SKUs. In light of these challenges, the conventional approach in the multivitamin market is the “one-size-fits-most” approach of targeting of a generically crafted formula to fit a large target broad group of people (like children, old women, etc.).

An alternative approach involves making customized multivitamins using a made-to-order approach. This sort of “bespoke” manufacturing is also logistically impractical, cost prohibitive, and practically impossible for a different set of reasons. To manufacture a single bottle/unit of such multivitamins can cost hundreds (perhaps even a thousand) dollars a bottle to make. To select the ingredients, accurately measure, weigh, mix, fill and lab test a single bottle of unique bespoke multivitamins is practically cost prohibitive for the typical consumer.

For example, to accurately measure out to the level of micrograms and mix as many as 50 ingredients at the level as little as one part per thousand would require labor, tools, and machinery not currently feasible for limited production runs of one individualized bottle/unit at a time. Additionally, the required post manufacturing diagnostic lab testing to ensure potency, purity, and accuracy, and to ensure the ingredients legally matches the label could cost as much as a thousand dollars due to the need to use expensive spectrography equipment and costly lab technician labor needed to do these tests. These lab tests, when done on a manufacturing production run batch of say 10,000 bottles/units, can result in a testing cost of ten cents per bottle. However, if each single bottle/unit must be tested individually, then each bottle alone must carry the $1,000 testing cost. Such bespoke multivitamins are thus practically infeasible.

The personalization of supplement regimens has historically required users to assemble individual supplement ingredients that the user self-administers in some sort of do-it-yourself routine. Historically, the set of ingredients that an individual selects for consumption comes from do-it-yourself research. This set of ingredients may be formed from a combination of recommendations, articles, doctor's advice, word-of-mouth, and/or marketing efforts. The user will then shop for each individual ingredients from various stores to assemble their personalized vitamin regime. This typically results in a cupboard, drawer, or box containing a set of individual supplement bottles/units that the user consumes based on their routine. This whole process requires a lot of self-education to determine the right regimen to follow and it is very time consuming to execute.

Additionally, building a personalized regime out of individual supplements is more expensive than if those same ingredients were put together in a single multivitamin. For example, if you were to consume 10 supplement ingredients in the form of 10 individual pills, it would be roughly 10 times more expensive than if those 10 ingredients were combined into a single multivitamin. This is because the costs of encapsulation and packaging typically account for most of a supplement's cost, not the raw materials themselves. Lastly, the do-it-yourself personalizer must take many pills a day compared to the convenience of taking a single multivitamin. If, for example, the do-it-yourself personalizer determines they need 20 ingredients for their optimal regime, they may be taking 20-40 individual pills a day (depending on dosage requirements). In contrast, multivitamins are a cheaper and easier way to take a lengthy list of supplements. As a result, many vitamin takers are forced to compromise or trade off the personalization advantages for the convenience and cost advantages of generic multivitamins.

Some approaches allow users to answer a survey or quiz to determine some of the users' general health histories. Examples of such questions can include the delineation of sex, weight, age, and possibly some general health background questions (e.g., hypertension, diabetes, etc.). With the collected information, a recommended set of individual vitamins to take are determined and those individual supplement pills are placed into a packet (or envelope) containing approximately 8-10 pills. However, with only 10 or so ingredients (in the form of individual pills) to work with (even in any combination), the user is only able to address a limited number of health conditions. For example, if a user had more than 10 health conditions that they would like to address, they would be left with many of those conditions unaddressed, and those that were addressed may only be addressed with a single ingredient. This common approach must ignore a large proportion of conditions, and typically can only recommend a single ingredient for each of the conditions that are treated. For example, if the approach puts 10 ingredients toward a condition such as hypertension, then it must ignore other conditions of concern.

In practice, such approaches typically recommend the six or seven most commonly missing nutrients in the average diet and then add three or four ingredients to the packet designed to help with one or more of the conditions of concern. The level of customization is therefore minimal. Additionally, due to economic and manufacturing machinery limitations, these approaches generally draw from a small palette of possible supplements (e.g., pills in their warehouses) resulting in a limited variety of recommended combinations. Because of these operational limitations, the number of ailments these organizations can help alleviate is quite limited. Furthermore, the manufacturing costs associated with selecting and filling individual envelopes vs. traditional packaging approaches (e.g., pills in a bottle) make it a relatively expensive manufacturing process for packaging supplements. Finally, unlike a true multivitamin, it would be economically infeasible to scale the personalization of this model to, for example, 40 or 60 ingredients in four to six packets a day. Such a solution could cost $500 a month and it would be extremely challenging for a consumer to take multiple packets containing 40-60 pills a day.

It should further be noted that these packets of vitamins are not multivitamins, but assemblages of individual supplement pills. Thus, they are subject to the same consumer cost and consumption disadvantages (vs. multivitamins) associated with taking individual pills as in the case of the do-it-yourself personalization approach.

The embodiments described herein can address one or more of the above-described challenges and therefore represent a significant technical advancement in the art. Embodiments described herein can provide personalized multivitamins to a user via a process that is economically feasible and that is capable of implementation using realistic warehouse space and manufacturing operations. Moreover, the embodiments provide more precise and more robust customization of supplements (to the level of personalization) to enable better health outcomes for the user. Better outcomes may include, for example, better management of health conditions, better avoidance or delay of health risks, and/or better ability to reach health goals.

In addition, because the personalized multivitamins are provided as a reasonable number of pills (and/or other dosage forms) per day, the user is better able to achieve compliance with the recommendation. As compared to a do-it-yourself approach or an approach that provides a semi-customized packet of pills, the disclosed approach makes it easier to comply by (1) requiring a smaller number of pills (and/or other dosage forms) to taker per day, and (2) better allowing the user to form a long-term habit/routine. For example, assuming a scenario where a user has 30 targeted health conditions (requiring 1 to 3 ingredients per health condition), the disclosed embodiments can address those conditions with as few as 2 doses per day, whereas a do-it-yourself approach or a semi-customized packet of pills approach would require daily consumption of roughly 30 to 90 different pills, each pill containing a specific single supplement ingredient. Beyond simply being more difficult to administer so many pills per day, it is also difficult to maintain such a routine long enough to establish a habit that can accomplish desired long-term outcomes. With the do-it-yourself approach in particular, different ingredients will run out at different times, and management of the separate ingredients and their separate bottles/containers quickly becomes daunting to the average consumer.

The disclosed embodiments, as will be discussed in more detail later, capitalize on a combination of three different methodologies or principles to create a synergistic effect that has been unachievable until now. These methodologies include (1) a technique focused on unique “combinatorial daily dosing” of multivitamins (e.g., dividing the assigned daily dosage of supplement ingredients into multiple parts, such as a morning set and an evening set, as opposed to one single dosage form per profile), (2) a technique focused on “intelligent formulation” of prepackaged units containing the supplement ingredients (e.g., multi-purpose over-formulation of multivitamins by over-engineering a blend or formula of supplements so that the resulting blend can simultaneously address multiple therapeutic conditions and targets), and (3) a technique focused on “dynamic personalization” (e.g., supplement labelling variations can be achieved via the intelligent acquiring or articulation of relevant personal data, and the labels can be customized to emphasize specific correlations between therapeutic conditions associated with the user and the selected supplements).

### Abbreviated List of Defined Terms

The following list of terms is not intended to represent an exhaustive glossary of all terms utilized in the disclosure. Terms not specifically defined herein may be given their ordinary meaning as understood by one of ordinary skill in the art. Moreover, the following definitions are intended to provide ease of reference and are not intended to specifically limit or narrow these terms any more than the ordinary meanings would suggest to the person of ordinary skill in the art.

The terms “supplement,” “multivitamin,” and their related terms are used interchangeably herein to refer to the tangible product made available to a user. For example, a “supplement” may refer to an entire prepackaged unit (e.g., a bottle, pouch, etc.) that contains a plurality of dosage forms (e.g., pills, capsules, tablets, etc.). More typically, a “supplement” refers to a dosage form itself. That is, a single pill may be referred to as a “supplement” or “multivitamin.” Where particular dosage forms such as pills are specified, it will be understood that other dosage forms are also applicable unless specified otherwise.

The term “supplement ingredients” and similar terms refer to the particular ingredients that may be combined within a dosage form to form the supplement. For example, zinc is a supplement ingredient, vitamin C is a supplement ingredient, echinacea is a supplement ingredient, and so on. The combination/blend of multiple supplement ingredients forms the overall “supplement.”

A “dosage form” is the individual unit that the user consumes, and may be a pill, tablet, capsule, or the like. Dosage forms are not necessarily limited to self-contained units such as capsules or tablets, although such are typical. For example, some embodiments may include powders, tinctures, or the like. These dosage forms may be associated with a measuring device such as a scooper or dropper to enable the user to readily measure and provide one or more unit doses.

“Personalized” supplements are those that have a level of customization sufficient to be substantially unique to the individual, or at least unique to the individual and others with substantially similar health characteristics and/or histories. This is used in contrast to conventional “customized” supplements which are only customized with respect to very broad categories of people (e.g., men's formula, women's formula, old age formula). The embodiments described herein do make use of customization, but the customization is sufficiently granular to generate “personalized” supplements.

As used herein, the phrase “health profile elements” refers to any type of health problem (e.g., diabetes, hypertension, sleep apnea, etc.) the user may have, any type of risk factor for a potential health problem the user may be at risk for or be concerned about (e.g., based on a family history of heart disease, family history of brittle bones, etc.), and/or any type of health goal (e.g., a goal to improve sleep, a goal to reduce stress, etc.) the user may have. Thus, the phrase “health profile element” should be interpreted broadly as including an expansive set of information related to the user's health, where that information can be obtained from different sources.

As used herein, the term “profile” refers to a collection of data (e.g., the user's health profile elements) describing the user as well as a collection of data describing what supplement ingredients the user may be recommended. That data can include the information about the user's health profile elements, such as the user's specific health problems, risk factors, and/or health goals. The data can further include information obtained from other sources, such as perhaps a health history maintained by a medical professional. The profile can further include a listing of therapeutic targets that are identified based on the user's health profile elements. The profile can further include a listing of dietary supplement ingredients that can be used to help address the therapeutic targets.

A “therapeutic target” is a subset of possible “therapeutic conditions,” identified based on the user's health profile elements, that may be addressed for that user. The term “therapeutic target” is thus synonymous with phrases such as “identified therapeutic conditions” or “therapeutic conditions associated with the user.” Therapeutic targets have a relationship with raw profile data (e.g., they can represent the profile data points from the raw data, which is actionable). Thus, one or more “therapeutic targets” may be selected from a set of “therapeutic conditions” based on the user's health profile elements.

### Example Systems and Methods

Embodiments disclosed herein relate to systems, devices, and methods for personalizing dietary supplement ingredients for multiple profiles and for personalizing dietary supplement ingredients for a specific user.

Some embodiments receive information detailing various health profile elements for the user. From the information detailing the health profile elements of the user, the embodiments can then identify specific therapeutic targets, from a set of therapeutic conditions, to associate with the user's health profile elements. Identifying a therapeutic condition as a therapeutic target then provides a pathway for the embodiments to address the user's health profile element.

In some instances, although the user might not actually have a specific therapeutic condition, a therapeutic condition can be identified as a therapeutic target of the user. For instance, the user's health profile elements may indicate that the user has a goal to improve his/her sleep. Insomnia is an actual therapeutic condition related to sleep. Treatments that would otherwise be used to address insomnia can also be used to help satisfy the user's goal of improving his/her sleep. Therefore, although the user may not have insomnia, the embodiments can correlate or associate insomnia with the user because the treatment for insomnia can be used to address the user's stated health profile element.

As another example, the user's health profile elements may indicate that the user has a health risk factor for diabetes. Here, the user only has a risk factor for diabetes, but the user does not actually have diabetes. Nevertheless, the embodiments can identify diabetes as a therapeutic target to address the user's risk factor. The supplemental treatments for diabetes can be used to address the user's risk factors, as outlined in his/her health profile elements. Accordingly, the health profile elements (e.g., the user's health problems, risk factors, and/or health goals) can be analyzed by the embodiments, and a corresponding set of one or more therapeutic targets can be identified.

The embodiments also access a data structure that maps various therapeutic conditions to dietary supplement ingredients designed to alleviate or treat those therapeutic conditions. The mapping can be consulted to identify which dietary supplement ingredients can be used to treat the identified therapeutic targets. Therefore, by treating the identified therapeutic targets with the various, corresponding dietary supplement ingredients, the embodiments address the user's health profile elements, which can include specific health problems, risk factors, and/or health goals. The dietary supplement ingredients are organized into bottles, containers, or other prepackaged units with corresponding SKUs (stock keeping units).

The embodiments identify a limited number of different types of prepackaged units (i.e., SKUs) of dietary supplement ingredients. The selected SKUs include a mixture of supplement ingredients that can be used to address the identified therapeutic targets, which are identified based on the user's health profile elements. The embodiments select a set of at least two prepackaged units. The set of prepackaged units constitutes a divided daily dosage for the user that includes dietary supplement ingredients designed to address the therapeutic targets of the user.

The formulas of the prepackaged units can be intelligently formulated. For example, a “basic” set of prepackaged units can be designed to be multi-purposefully over-formulated such that they contain a set of supplement ingredients that can address multiple different sets of therapeutic conditions. This allows the basic prepackaged units to be used even for user profiles that are relatively rare (e.g., that contain a rare set of therapeutic targets). Additional “advanced” prepackaged units can be formulated with supplement ingredients that better focus on certain sets of therapeutic conditions. For example, certain sets of therapeutic conditions may be common among the user base, and a set of prepackaged units with an advanced formula tailored to better focus on such a condition set can minimize the need to use an over-formulated set of prepackaged units for those users. That is, for a user with a set of therapeutic targets that corresponds to an advanced formula, the embodiments can assign a set of prepackaged units with an advanced formula that better focuses on that user's needs. At the same time, a user with a relatively rare set of therapeutic conditions can still be assisted with a more basic (i.e., more over-formulated) formula. This concept avoids the need to generate formulas specific to very rare user profiles (which would raise costs by requiring the generation of SKUs that are rarely, if ever used), while at the same time allowing for the generation of more focused formulas when there is sufficient user base to justify the creation of the corresponding prepackaged units and SKUs.

Machine learning techniques may be utilized to analyze the set of user profiles and assist in determining advanced formulas to better address a sufficient subset of the user base. For example, as additional user profiles are generated, machine learning techniques can identify subgroups with similar profiles (i.e., similar sets of therapeutic targets). When sufficient numbers of such users exist, a formulation can be generated that better focuses on the particular therapeutic targets of this subgroup of users. Formulations can better focus on such user profile subgroups by removing supplement ingredients that are not useful to the subgroup, adding supplement ingredients that are useful to the subgroup, and/or or increasing the potency of supplement ingredients that are useful to the subgroup, for example.

The embodiments can also generate Supplement Facts labels for the set of prepackaged units. These labels are designed to identify correlations between the dietary supplement ingredients included in the set and the therapeutic targets specific for that user. That is, the embodiments capitalize on the fact that a single supplement ingredient can be used to address multiple therapeutic conditions. The embodiments can dynamically tailor the labels to emphasize the correlation between the user's supplement ingredients and the specific therapeutic target(s) of the user. Therefore, despite a particular dietary supplement ingredient being usable to address multiple different therapeutic conditions, the labels are designed to illustrate and emphasize the correlations that are relevant to the specific therapeutic targets of the user. These correlations can be illustrated on the generated Supplement Facts label as a listed set of proprietary blends. The blend names (e.g., immunity blend, athletic performance blend, cognition blend, etc.) represent the therapeutic targets of the user or generalized categories of the therapeutic targets of the user.

### Examples Of Technical Benefits, Improvements, And Practical Applications

The following section outlines some example technical improvements and practical applications provided by the disclosed embodiments. It will be appreciated, however, that these are just examples and that the embodiments are not limited to only these improvements.

Whereas historical methods for generating multivitamins were limited to consideration of only one or perhaps a small number of “variables” (e.g., less than 3), the disclosed embodiments are configured to allow the consideration of an almost unlimited number of variables to generate multivitamins in a cost-effective manner.

As used herein, the term “variables” can include any type of characteristic related to a user. Examples of such characteristics can include a user's biological/demographic factors (e.g., age, weight, height, sex, etc.). Another example of a characteristic can include the user's health risk factors, such as the user's current health-related conditions, family medical history, or potential future health related risks to which the user may be subjected. The variables can also include goals for the user. A variable can also be used to describe the user's lifestyle, such as whether the user leads an active or sedentary lifestyle. Indeed, a variable can be any type of metric associated with a user and can be used as a data point within the user's profile.

The disclosed embodiments can generate multivitamins for an almost unlimited number of different “classes” or “groups” of users. As used herein, these terms generally refer to one or more individuals who share a common set of one or more different characteristics (e.g., variables). It is typically the case that each user will have his/her own specific profile, though it is possible that very similar users may share the same profile. Notably, the embodiments are configured to enable the generation of personalized multivitamins for any number of different users, or rather user profiles. Inasmuch as there are billions of people on the earth, the embodiments can generate personalized multivitamins for billions of unique profiles.

As an example, a first profile or class of individuals may have 10 characteristics or “variables” in common (e.g., female, 60-65 age range, iron deficient, vitamin D deficient, pre-diabetic, hypertension, sedentary lifestyle, calcium deficient, vitamin K deficient, and high salt intake). A second profile or class of individuals may have 6 characteristics in common. The disclosed embodiments are able to beneficially personalize a set of multivitamins for the first profile and are able to personalize a set of different multivitamins for the second profile in a cost-effective manner. Indeed, the embodiments can personalize multivitamins for essentially an unlimited number of different profiles.

The disclosed embodiments also optionally incorporate the use of machine learning and feedback. For example, the embodiments can use feedback obtained from a large set of users and provide that feedback as input into a machine learning engine. The machine learning engine can then perform a meta-analysis on the impact of the supplements. Furthermore, this analysis can be performed on hundreds or thousands of different supplements. While doctors are highly intelligent, they cannot keep this amount of information or this level of granularity at the forefront of their minds. Doctors can also be subject to inadvertent bias. The embodiments can beneficially rely on databases, algorithms, and/or machine learning to make inferences and to improve mappings between supplements and therapeutic conditions without bias.

Such machine learning and/or other artificial intelligence (AI) tools may be integrated with the disclosed embodiments. For example, AI-driven natural language models may be utilized to interact with the user for gathering health profile information via a survey. The survey can identify known health issues and can also uncover previously unknown health risks or conditions. An AI-driven survey that leverages natural language query/response models can also more intelligently pursue relevant lines of questioning while better ignoring irrelevant issues and/or lines of questioning that the user has shown are of limited concern or interest. Dynamic adjustments to the presentation of questions can be adjusted “on the fly”, such as by omitting categories or questions determined to be of no interest or that are causing confusion to the user, while supporting categories or questions determined to have relevance and/or to be of interest to the user.

An AI-driven survey can also improve the speed of answer processing. In addition, an AI-driven survey can be configured to monitor not only the user's explicit answers, but other contextual indicators such as user answers within the context of past answers, answer options that are ignored, dwell time on individual questions/pages, and other oblique indicators of a user's interest.

### Example Architecture

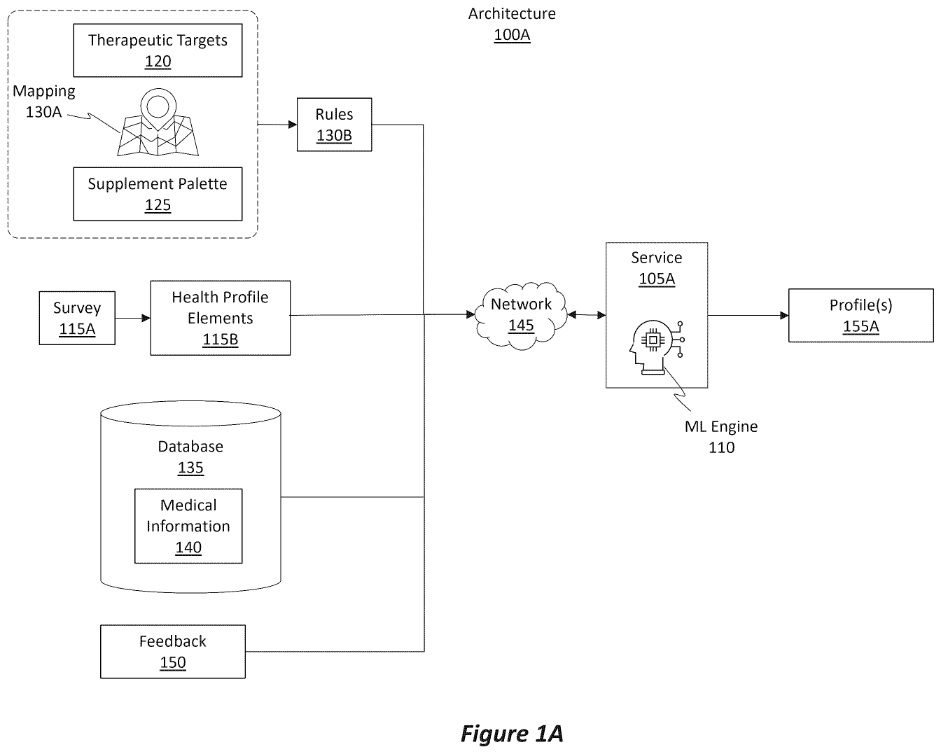

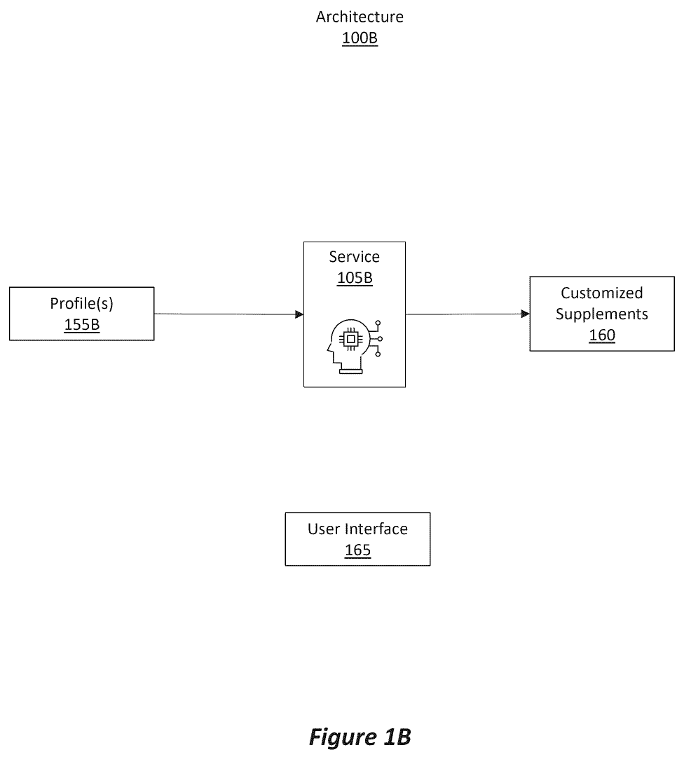

Having described some of the high-level benefits provided by the disclosed embodiments, attention will now be directed to FIGS. 1A and 1B. FIG. 1A illustrates an example architecture 100A that can be used to achieve the disclosed benefits. Architecture 100A is shown as including a service 105A. The service 105A can be a cloud computing service, such as a software as a service (SaaS) type of a service. In some cases, the service 105A can also be a locally instantiated service operating on a local device.

In some optional cases, the service 105A can include or make use of a machine learning (ML) engine 110 that can carry out any of the machine learning implementations described herein. As used herein, “machine learning” may include any type of machine learning algorithm or device, convolutional neural network(s), multilayer neural network(s), recursive neural network(s), deep neural network(s), decision tree model(s) (e.g., decision trees, random forests, and gradient boosted trees) linear regression model(s), logistic regression model(s), support vector machine(s) (“SVM”), artificial intelligence device(s), or any other type of intelligent computing system. Any amount of training data may be used (and perhaps later refined) to train the machine learning algorithm to perform the disclosed operations. Further details on some of the functionality of the ML engine 110 will be provided later, such as how the ML engine 110 is able to modify a mapping data structure based on user feedback.

The service 105A is designed to receive information about a user and to generate a unique profile for that user. This profile includes information regarding what dietary supplements the user should take to help improve the user's health and/or lifestyle. Notably, multiple sources can be used to feed information into the profile such that the profile is not simply limited to information provided by a user. For example, the profile can be sourced from medical repositories, other profiles, blood test data, and/or any other affiliate, and the profile can optionally be managed by the user. As the user's health evolves, the user can update his/her profile to reflect these changes.

To generate a profile, the service 105A can acquire various pieces of data and is further able to access various pieces of data. To illustrate, the service 105A can facilitate a survey 115A, which the user can complete. The survey 115A acquires information about the user's health profile elements 115B. As mentioned earlier, these health profile elements 115B can include specific health problems the user is currently facing and/or risk factors the user may currently be facing or may later face (e.g., a family history of a certain disease or ailment), and/or various health related goals the user may have (e.g., improve endurance running or stamina).

The service 105A is also able to acquire information concerning a set of therapeutic conditions and generate a set of therapeutic targets 120 therefrom. Examples of therapeutic conditions include actual diseases or diagnosable ailments a human may face. As examples only, the therapeutic conditions can include hypertension, diabetes, heart disease, and so on.

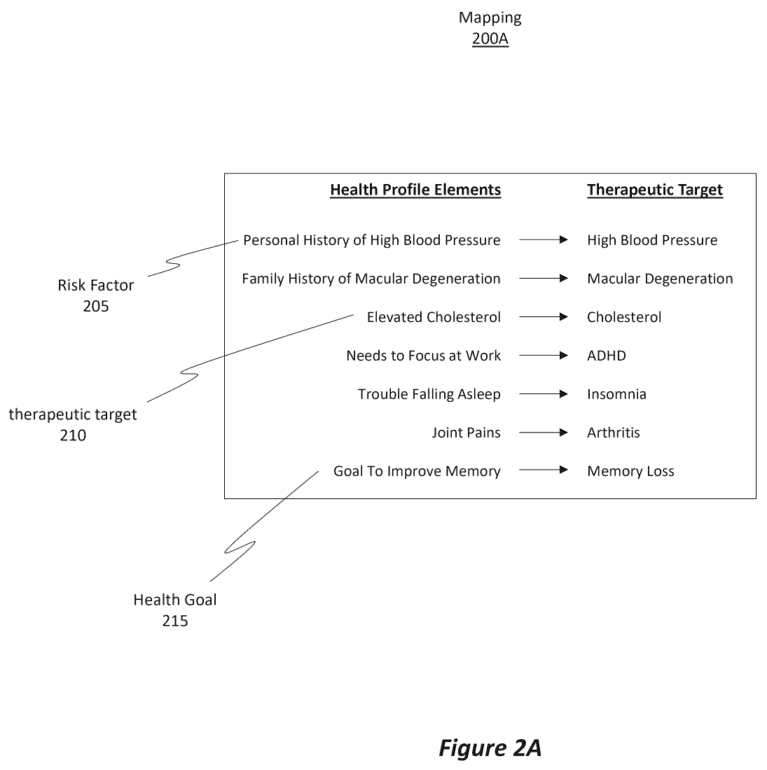

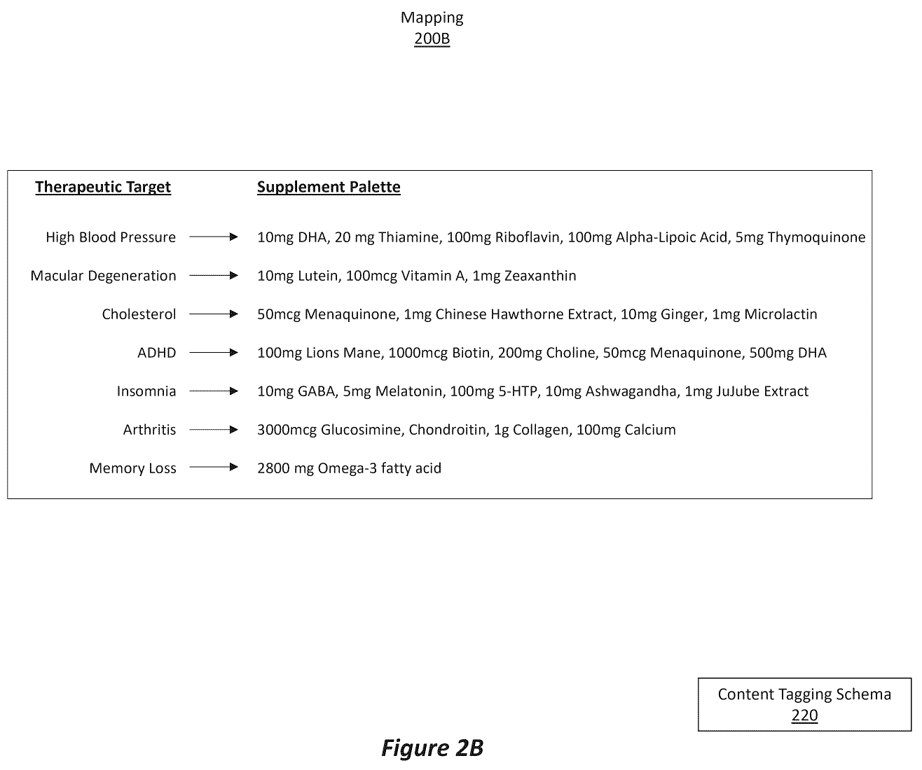

The service 105A is able to generate and/or access a supplement palette 125 that includes any number of dietary supplements. The service 105A can further generate and/or access a mapping 130A that links specific supplements to specific therapeutic conditions to thereby identify the therapeutic targets 120 of the user. FIGS. 2A and 2B are illustrative of an example mapping.

FIG. 2A shows a mapping 200A that is representative of the mapping 130A from FIG. 1A. Notice, the mapping 200A first identifies correlations between various health profile elements and actual therapeutic conditions, thus identifying the user's therapeutic targets. To illustrate, FIG. 2A shows one example of a health profile element in the form of a risk factor 205, which is listed as being a “Personal History of High Blood Pressure.” Another example of a health profile element is in the form of a health problem 210 (e.g., elevated cholesterol). Yet another example of a health profile element is in the form of a health goal 215 (e.g., a goal to improve memory).

In accordance with the disclosed principles, the embodiments can intelligently associate or correlate a health profile element with an actual, addressable therapeutic target. For instance, the risk factor 205 (i.e., the personal history of high blood pressure) is associated with the therapeutic target of high blood pressure. The health problem 210 (i.e., the elevated cholesterol) is associated with the therapeutic target of cholesterol. The health goal 215 (i.e., the goal to improve memory) is associated with the therapeutic target of memory loss. Thus, therapeutic conditions that are associated with one or more of the user's health profile elements define the user's therapeutic targets. Supplements that are usable to address the therapeutic target can be used to address the user's health profile element. For instance, a supplement designed to help with memory loss will also advance the goal of improving one's memory.

According to the operations disclosed herein, the service 105A is beneficially able to generate a user profile that is robust and granular. A robust and granular health profile is unhelpful, however, if it cannot be effectively utilized to provide dietary supplement ingredients at the same or similar level of granularity. For example, a robust and granular health profile has limited utility if the corresponding mapping to dietary supplement ingredients provides a relatively limited number of ingredient options. Here, the user profile is meaningful and practically implementable due to the robust mapping between health profile elements and therapeutic conditions, and between therapeutic conditions and corresponding recommended supplement ingredients. The ability of the user to access, update, monitor, and/or adjust his/her health profile further adds a level of control that enables even more user engagement and awareness.

FIG. 2B shows how the user's specific therapeutic targets can be linked with specific dietary supplement ingredients that, when taken, can address, treat, or help alleviate the therapeutic target. To illustrate, for the therapeutic target of high blood pressure, the following set of supplement ingredients can be taken, in a “recommended daily dose” to help alleviate that condition: 10 mg of DHA, 20 mg of Thiamine, 100 mg of Riboflavin, 100 mg of Alpha-Lipoic Acid, and 5 mg of Thymoquinone. For the therapeutic target of cholesterol, the following set of supplement ingredients have been shown to help: 50 mcg Menaquinone, 1 mg Chinese Hawthorne Extract, 10 mg Ginger, 1 mg Microlactin. For the therapeutic target of memory loss, the following set of supplement ingredients have been shown to help: 2800 mg Omega-3 fatty acid. Thus, supplement ingredients can be linked with therapeutic targets, which are identified from the user's health profile elements.

The different supplement ingredients can be tagged or can have metadata associated with them in a database to indicate conditions and/or condition types that they can be used against. That is, some embodiments use a content tagging schema 220 to tag supplement ingredients with auxiliary information to assist in mapping those supplement ingredients to associated conditions.

Reviewing the mappings 200A and 200B, one can observe that some supplement ingredients can address multiple different therapeutic conditions. This recognition allows the embodiments to use a limited number of blends or formulas of supplement ingredients to address a wide variety of different therapeutic targets, as described in more detail below.

Returning to FIG. 1A, the service 105A is able to access or generate the mapping 130A. Additionally, the service 105A is able to generate a set of rules 130B based on the mapping 130A, where these rules 130B reflect the mapping 130A and reflect which supplement ingredients can be used to address which therapeutic targets.

In some cases, the service 105A can acquire health information about the user from a variety of sources. For instance, the service 105A can acquire information from a database 135 comprising medical information 140 about the user (subject to authorization from the user and in accordance with applicable health privacy rules). The user may authorize the service 105A to acquire the user's health history from his/her medical doctors and/or other entities.

In some cases, these various inputs can be received over a network 145, such as a connection via the Internet. In some cases, the information can be provided directly to the service 105A. The service 105A compiles the acquired information and uses the information to generate a profile for a user, as shown by profile(s) 155A.

FIG. 1B shows an architecture 100B, which is an extension of architecture 100A from FIG. 1A. The architecture 100B shows a service 105B, which can optionally be the same service as service 105A and/or can be a new service. The service 105B reviews the generated profile(s) 155B, which are representative of the profile(s) 155A from FIG. 1A. Based on the profile(s) 155B, the service 105B selects a set of personalized supplement ingredients 160 for the user, where the supplements in the personalized supplements 160 are designed to address the therapeutic targets of the user.

It should be noted that the user can optionally manage his/her own profile, such as via a user interface 165. The user can add data, remove data, and/or manage the data in any other manner. In this sense, the user profile is manageable by the user in at least some aspects. Such management is particularly beneficial because the user's health will likely change as he/she consumes the personalized supplement ingredients.

Returning to FIG. 1A, as will be described in more detail later, the service 105A can also receive feedback 150 from a user regarding the efficacy of his/her personalized supplement ingredients. In some cases, the ML engine 110 can use the feedback 150 to modify the mapping 130A to improve how combinations of supplements alleviate therapeutic conditions.

### Example Process Flow

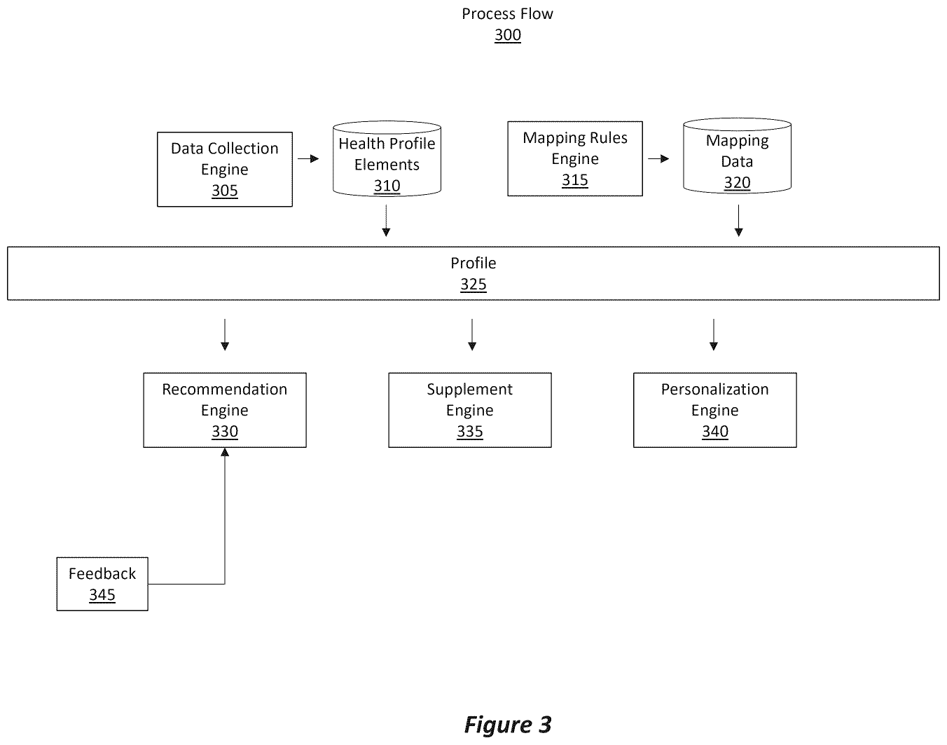

Attention will now be directed to FIG. 3, which illustrates an example process flow 300 that can be implemented by the services 105A and/or 105B of FIGS. 1A and 1B. FIG. 3 shows various so-called “engines” that can perform different operations. One will appreciate how these engines can be sub-components of the services 105A/105B. Also, it should be noted how some of these various processes can be performed in parallel with one another. Therefore, unless explicitly stated otherwise, there may not be a temporal dependency or linkage between the various processes described in FIG. 3.

In some embodiments, a data collection engine 305 can be used to collect and store information about health profile elements 310 for the user. Information about the health profile elements 310 can be acquired from a survey, from a medical database, or via any other suitable means. From the information about the user's health profile elements 310, the embodiments can identify therapeutic targets, as was described in relation to FIG. 2A. Thereafter, the embodiments can identify specific supplement ingredients that can be used to alleviate the therapeutic targets, as was described in relation to FIG. 2B.

The mapping rules engine 315 can consider information on therapeutic conditions as well as on supplement ingredients. Any type of research document or publication can be queried to identify which supplement ingredients can be used to address which therapeutic conditions. The mapping data 320 reflects a generated mapping (e.g., mapping 130A from FIG. 1A) and/or rules (e.g., rules 130B) that identify links between therapeutic conditions and supplements.

The health profile elements 310 and even the mapping data 320 can be used by the service to generate a profile 325 for the user. Inasmuch as every human is unique and has unique characteristics, it will likely be the case that no two profiles are exactly the same. With the profile 325 populated with the user's health profile elements 310 and the mapping data 320, the service can then identify specific therapeutic conditions to associate with the user as therapeutic targets. Each of these may be identified because the user has the therapeutic condition, because the user is at risk for the therapeutic condition, or because the user has a goal related to the therapeutic condition.

The service includes a recommendation engine 330, a supplement engine 335, and a personalization engine 340. The recommendation engine 330 analyzes the user's profile, including the therapeutic targets of the user, to then recommend a set of supplement ingredients to address the user's therapeutic targets. The supplement engine 335 selects a specific set of SKUs (stock keeping units) or other prepackaged units of dietary supplement ingredients based on the recommendation engine 330's recommendation. Practically speaking, there are a limited number of different SKUs in any product line. Despite this, the embodiments have designed these SKUs to address a large number of different profiles. The personalization engine 340 then generates a unique label for the SKUs.

As also illustrated, the recommendation engine 330 can receive feedback 345 from the users. The feedback 345 can describe the efficacy of a set of dietary supplement ingredients in relation to their ability to address therapeutic targets. The feedback 345 can be received from individual users and/or it can be received on a widescale basis. The recommendation engine 330 can optionally modify the mappings between therapeutic conditions and supplement ingredients based on the feedback.

### Combinatorial Daily Dosing

To achieve highly personalized multivitamins for a large number of unique profiles using a limited number of different combinations of SKUs, the embodiments may rely on three principles or methodologies that complement one another. The first principle is referred to as combinatorial daily dosing. Combinatorial daily dosing divides the recommended daily dose of dietary supplement ingredients across different SKUs or other prepackaged units. In some embodiments, the different SKUs or other prepackaged units are designed to be taken at different times of the day (e.g., one SKU includes a morning dose and a different SKU includes an evening dose). Stated differently, the embodiments provide a mechanism for delivering a user's recommended daily dose of supplement ingredients using multiple prepackaged units/SKUs. Notably, the mechanisms can be broken out in a manner such that the SKUs comprising the supplement ingredients can be combined in different ways to provide a combinatorial increase in number of profiles without requiring a corresponding increase in number of SKUs.

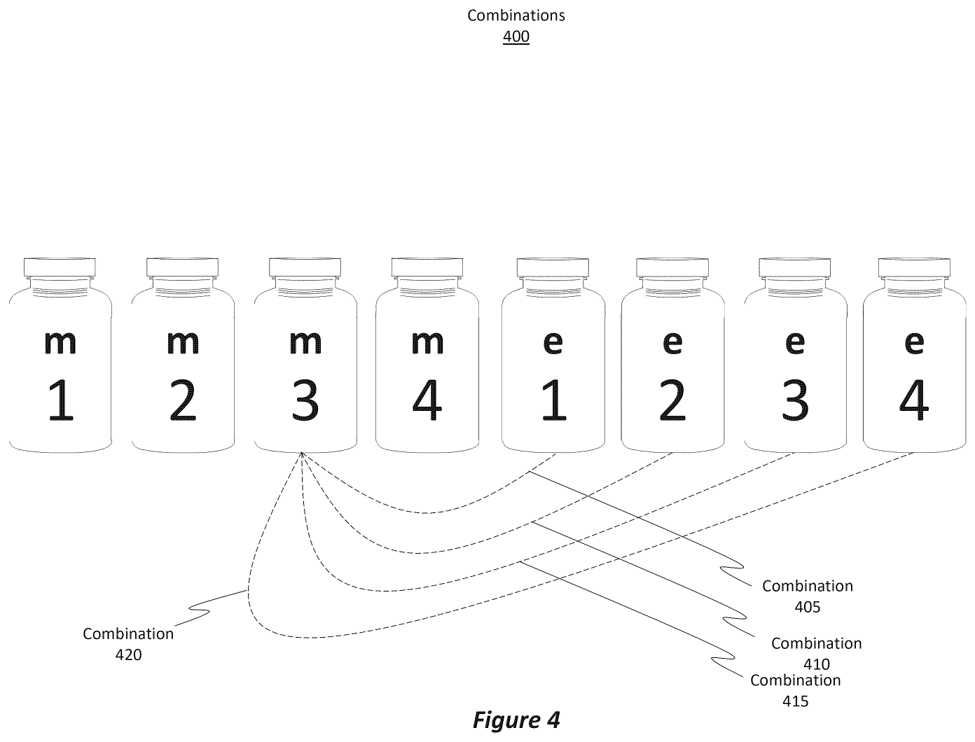

FIG. 4 illustrates a principle by which a user's recommended daily dose of supplement ingredients can be divided among multiple prepackaged units for distributed administration throughout the course of a day, as shown by combinations 400. In some cases, a first dosage can be consumed by the user during a morning period (e.g., the “m” on the illustrated bottles) and a second dosage can be consumed by the users during an evening period (e.g., the “e” on the illustrated bottles). Additional dosages can be consumed throughout the day as well, such as a mid-day/lunchtime dosage and/or some additional dosage (e.g., a supplemental gummy, soft gel, liquid gel, or some additional multivitamin at some other time period).

The combinations 400 include, but are not limited to, the illustrated combinations 405, 410, 415, and 420, each comprising one SKU for the morning and one SKU for the evening. The illustrated example includes the following morning bottles: an “m1” bottle, an “m2” bottle, an “m3” bottle, and an “m4” bottle and the following evening bottles: an “e1” bottle, an “e2” bottle, an “e3” bottle, and an “e4” bottle. In this example, there are 16 available combinations of morning and evening SKUs.

The “m1” and “e1” bottles can be considered as a set of multivitamins for a basic user profile (e.g., perhaps a profile that can be used to cover the conditions that are most common to the general population). The other combinations can include the same multivitamins as in the basic user profile combination, but with additional multivitamins or supplements to address additional therapeutic conditions. For instance, the “m2” and “e2” bottles may include supplements that include athlete-specific types of supplement ingredients or perhaps menopausal-related supplement ingredients. By distributing different types of supplements into different prepackaged units and by optionally having a user consume the supplements at different times of the day, the embodiments can provide multiple potential combinations of SKUs (and thus multiple different profiles) while still being cost conscientious. For instance, based on the user's profile, the embodiments may recommend the user take the “m1” and “e1” bottles. As another example, the embodiments may recommend the user take the “m1” and “e2” bottles, or perhaps the “m1” and “e3” bottles, or perhaps the “m4” and “e2” bottles, and so on, thereby leading to a larger number of SKU combinations than SKUs.

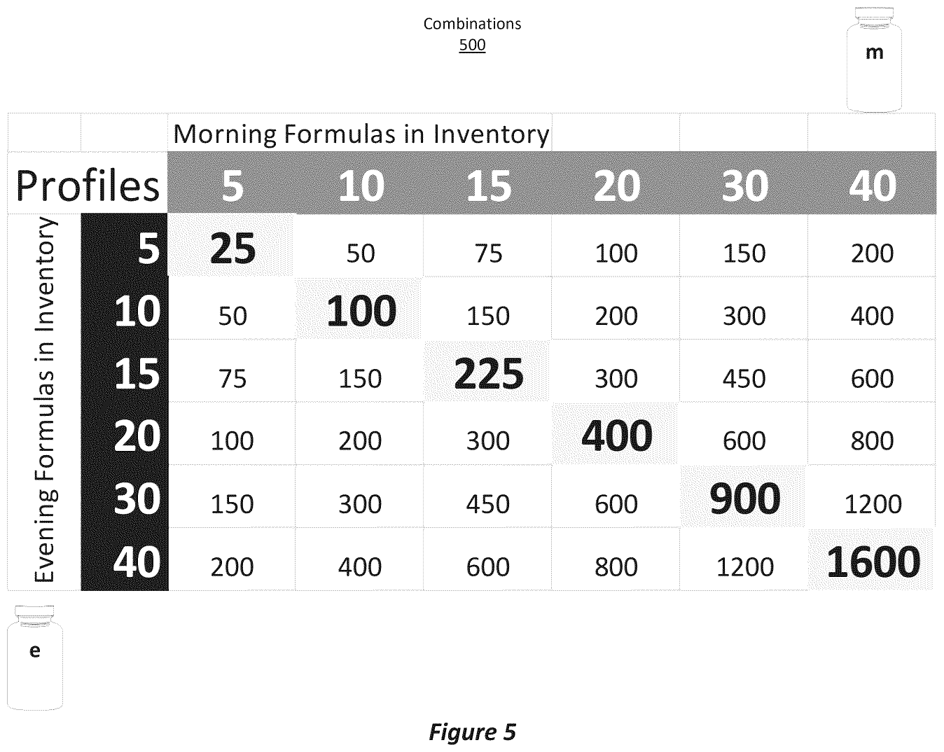

FIG. 5 shows a set of combinations 500, and illustrates the number of unique combinations can be formed from a limited number of SKUs. As an example, suppose there are 5 different blends or formulas available for morning consumption and 5 different blends or formulas available for evening consumption. From these 10 total SKUs, there is a possibility of generating 25 different combinations (each with 1 morning and 1 evening SKU). Now, consider a scenario where there are 40 different formulas available for morning consumption and 40 different formulas available for evening consumption. With these 80 total SKUs, there are 1,600 different combinations available.

Accordingly, by dividing the daily dosage for a user across different time periods of the day, and by enabling different combinations of SKUs to support different profiles, the embodiments are able to significantly increase the number of different classes and profiles that are supportable. High levels of personalization can be achieved without requiring impractical numbers of corresponding SKUs and associated inventory costs and warehouse management.

The embodiments can further increase the number of profiles that can be serviced by capitalizing on the notion of multi-purpose over-formulation and dynamic personalization.

### Intelligent Formulation

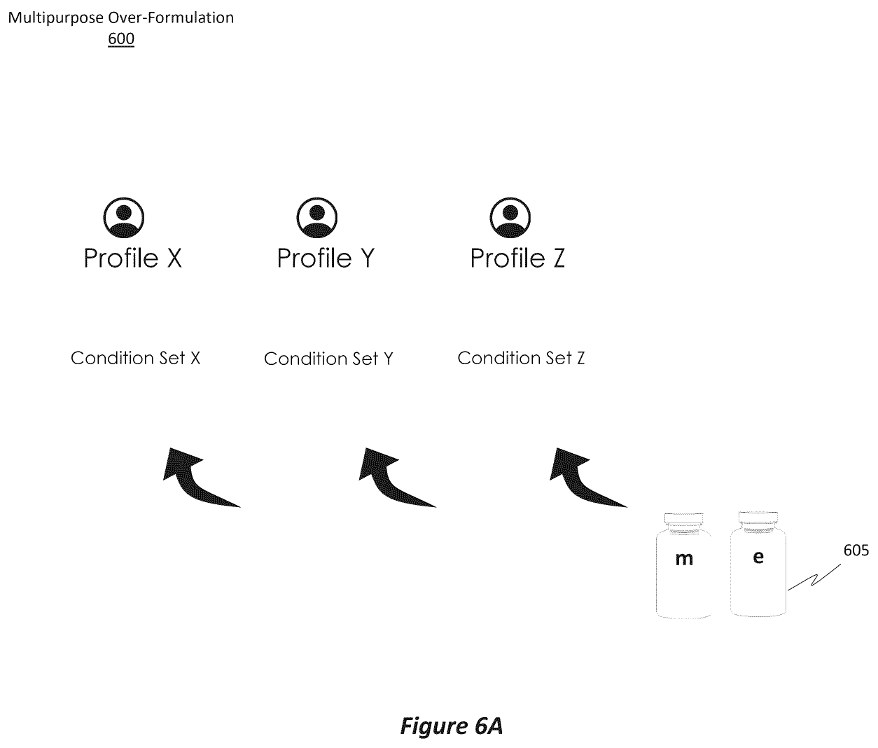

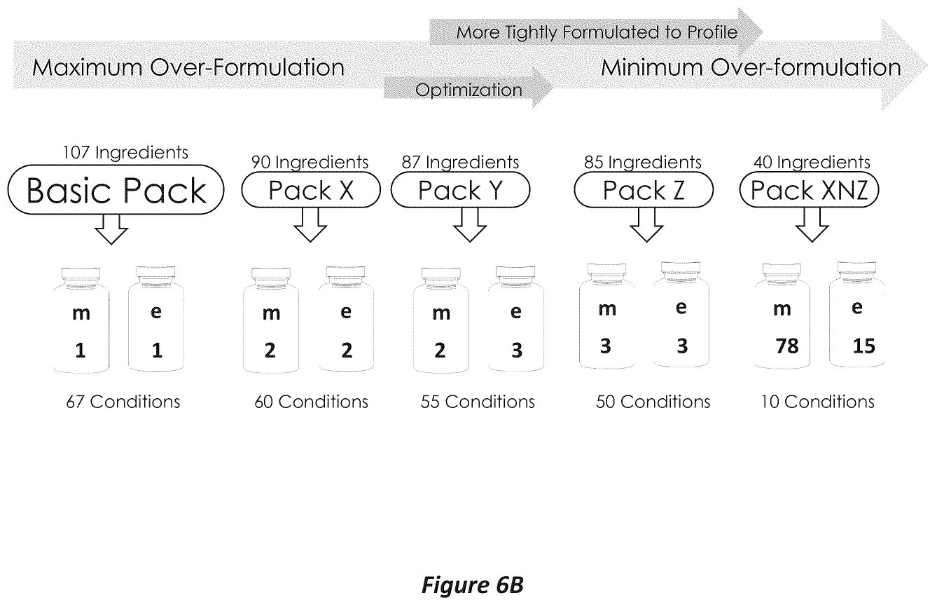

The embodiments can intelligently generate the formulations of the prepackaged units, and this principle can include multi-purpose over-formulation. With reference to FIG. 6A, multi-purpose over-formulation 600 refers to the notion that a formula blend of dietary supplements can be designed to address a large number of different therapeutic conditions. For example, the set of prepackaged units 605 can be suitable for addressing multiple different user profiles (X, Y, and Z) even though they are each associated with different sets of conditions.

Multi-purpose over-formulation greatly expands the number of profiles that can be serviced, even above the large number that could be serviced using combinatorial daily dosing alone. Further, a limited set of SKUs can be used to address many different targets. The concept of multi-purpose over-formulation therefore intelligently organizes supplement ingredients into different SKUs such that a minimal number of SKUs can service a large number of different therapeutic conditions and therefore a large number of user profiles.

FIG. 6B illustrates how the level of multi-purpose over-formulation can be intelligently adjusted across different formulas. A “basic” formula can, for example, be formulated to address a very broad set of therapeutic conditions, and such a formula may be included in the “m1” and/or “e1” SKUs, as in the illustrated example. This allows the basic prepackaged units to be used even for user profiles that are relatively rare (e.g., that contain a rare set of therapeutic targets). Additional “advanced” prepackaged units can be formulated with supplement ingredients that better focus on certain subsets of therapeutic conditions (see the progression to the right in FIG. 6B). For example, certain sets of therapeutic conditions may be common among the user base, and a set of prepackaged units with an advanced formula tailored to better focus on such a condition set can minimize the amount of over-formulation of the prepackaged units for those users. That is, for a user with a set of therapeutic targets that corresponds to an advanced formula, the embodiments can assign a set of prepackaged units with an advanced formula that better focuses on that user's needs. At the same time, a user with a relatively rare set of therapeutic conditions can still be assisted with a more basic (i.e., more over-formulated) formula. This concept avoids the need to generate formulas specific to very rare user profiles (which would raise costs by requiring the generation of SKUs that are rarely, if ever used), while at the same time allowing for the generation of more focused formulas when there is sufficient user base to justify the creation of the corresponding prepackaged units and SKUs.

More advanced formulations can better focus on user profile subgroups by removing supplement ingredients that are not useful to the subgroup, adding supplement ingredients that are useful to the subgroup, and/or or increasing the potency of supplement ingredients that are useful to the subgroup, for example. In some embodiments, the potency level of the supplements can be modified based on what type of profile they are supporting. Some potencies can be increased or decreased. In some cases, feedback can be used, and the ML engine can selectively modify the potency levels based on the feedback.

Machine learning techniques may additionally or alternatively be utilized to analyze the set of user profiles and assist in determining advanced formulas to better address a sufficient subset of the user base. For example, as additional user profiles are generated, machine learning techniques can identify subgroups with similar profiles (i.e., similar sets of therapeutic targets). When sufficient numbers of such users exist, a formulation can be generated that better focuses on the particular therapeutic targets of this subgroup of users.

The mapping 125 from FIG. 1 in combination with the ML engine 110 can be used to expand and/or modify which supplements can be used for which therapeutic conditions. This mapping 125 can be expanded over time as new research emerges and/or as new user data is obtained. To further increase the personalization of multivitamins, the embodiments thus rely on the notion that various supplement ingredients can have multiple purposes.

Accordingly, the combination of combinatorial daily dosing and intelligent formulation, including multi-purpose over-formulation, exponentially increases the number of serviceable profiles without requiring concomitant increases in the number of unique SKUs. In addition to these two principles, the embodiments may additionally utilize a third principle, dynamic personalization.

### Dynamic Personalization

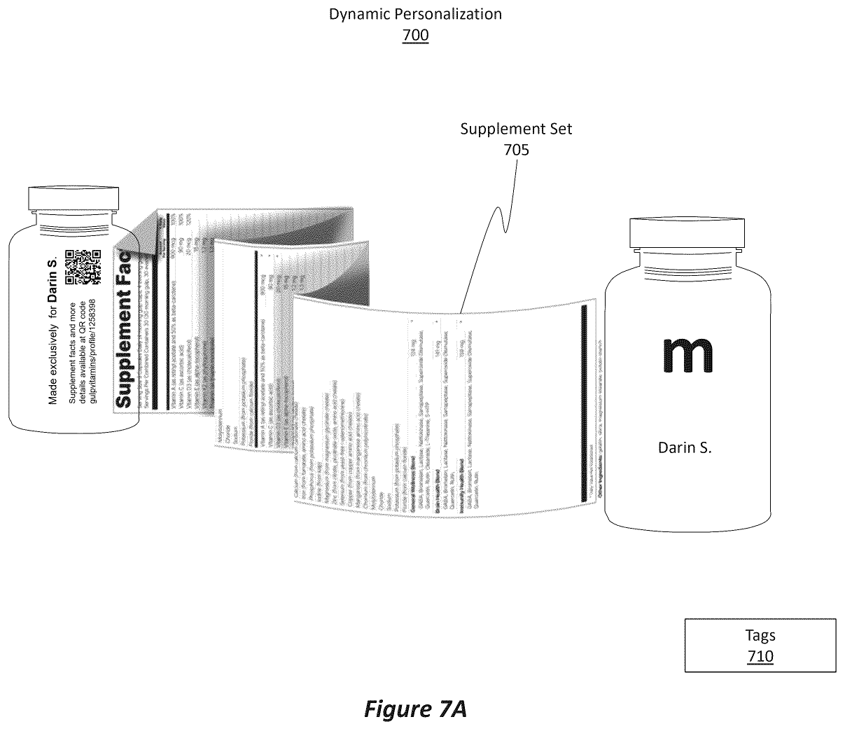

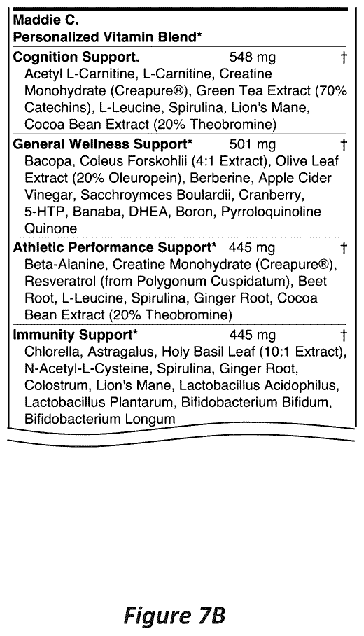

FIGS. 7A and 7B illustrate the notion of dynamic personalization 700. Dynamic personalization 700 enables Supplement Facts labels to be generated in real-time and to be configured to reflect a correlation between specific supplement ingredients and specific therapeutic targets of a particular user. These correlations can be reflected on the Supplement Facts panel as a list of proprietary blends relevant to the user. Each therapeutic target of the user can correspond to a blend indicated on the label.

FIG. 7A illustrates an SKU with a personalized supplement set 705. FIG. 7B illustrates a portion of an example personalized Supplement Facts label, showing a list of proprietary blends relevant to the user “Maddie C.” based on the user's specific therapeutic targets. FIG. 7B shows, for example, a “Cognition Support” blend, a “General Wellness Support” blend, an “Athletic Performance Support” blend, and an “Immunity Support” blend, which correspond to specific therapeutic targets of user Maddie C. Other blends can include Diet and Nutrition Support, Gut Health Support, Mood Support, Energy Support, Skin Support, Abdominal Discomfort Support, Calmness Support, and/or other blends relevant to a user's specific therapeutic targets. The “General Wellness Support” blend can function as a “catch all” for supplement ingredients that do not fit within a specific therapeutic target blend.

As an example of dynamic personalization of the Supplement Facts label, suppose a first user is having sleep issues. To support the sleep issues, the embodiments can select a set of SKUs comprising supplement ingredients that include zinc and magnesium. The embodiments can personalize this user's bottles (or other applicable prepackaged unit) with a Supplement Facts label that emphasizes the relationship of zinc and magnesium to sleep. For example, the user's Supplement Facts label can list a “Sleep Support” blend that lists zinc and magnesium among its ingredients.

In another example, suppose a second user is having immunity issues. To support the immunity issues, the embodiments can select a set of SKUs (possibly, but not necessarily, being the same set of SKUs as the first user) that includes zinc and magnesium. Whereas with the first user, the Supplement Facts label indicated that zinc and magnesium were provided as part of a “Sleep Support” blend to address sleep issues (specifically geared to the first user's particular therapeutic target), the bottles or other prepackaged units for the second user can be labeled with to emphasize the relationship of zinc and magnesium to immunity issues (specifically geared to the second user's therapeutic target). For example, the second user's Supplement Facts label can list an “Immunity Support” blend that lists zinc and magnesium among its ingredients. Note that a user with both sleep therapeutic targets and immunity therapeutic targets can have a Supplement Facts label that lists both a “Sleep Support” blend and an “Immunity Support” blend, with both blends comprising zinc and magnesium.

Thus, even though potentially a similar set of supplement ingredients and/or SKUs are used for the first user and for the second user, the labels on the bottles or other prepackaged units can be personalized using therapeutic target proprietary blends to reflect how the supplement ingredients correspond to the user's respective specific therapeutic targets. These labels can be generated in real-time and on demand. Accordingly, the embodiments are focused on various mechanisms that can deliver supplement ingredients to a wide range of potential user profiles using a manageable number of SKUs and in a manner that presents the supplement ingredient information to the user in a more personalized manner.

The embodiments beneficially recognize that a single supplement ingredient can be used for a variety of therapeutic conditions. The embodiments dynamically adjust or edit the therapeutic target proprietary blends on the Supplement Facts labels to achieve heightened levels of personalization without having to modify the formula itself. Instead, the embodiments can use the mapping mentioned previously, and modify the therapeutic target proprietary blends on the Supplement Facts labels accordingly to reflect how a particular supplement ingredient is being used to address a specific user's therapeutic targets.

As shown in FIG. 7A, the supplement set 705 is a personalized label that shows how specific supplement ingredients are provided to service the user's particular therapeutic targets. As mentioned previously, some embodiments rely on tags 710, which can be in the form of metadata for a particular supplement ingredient. The tags 710 can indicate what therapeutic conditions a particular supplement ingredient can be used to address. These tags 710 can then be used for the mapping process, such as by the ML engine. That is, each supplement ingredient in the data structure can be tagged based on a content tagging schema. These tags can then be referenced by the ML engine to make recommendations.

Supplement Facts panels typically require that ingredients be listed in order of largest to smallest quantity. To meet this requirement while still providing dynamic personalization, the embodiments can, for each supplement ingredient associated with multiple blend categories, equally allocate the total quantity of the supplement ingredient (e.g., per serving) among each blend category that includes the supplement ingredient. For example, FIG. 7B shows that spirulina is included in the Cognition Support, Athletic Performance Support, and Immunity Support blends. As an example, if the total amount of spirulina per serving for the entire formula were 90 mg, the 90 mg would be divided so that each of the Cognition Support, Athletic Performance Support, and Immunity Support blends include 30 mg of spirulina. After such allocation, the supplement ingredients of each blend can be listed in order of largest to smallest quantity, and the blends themselves can be totaled and listed in order of largest to smallest quantity.

Accordingly, the utilization of one or more of combinatorial daily dosing, multi-purpose over-formulation, and dynamic personalization enables effective personalization of multivitamins without requiring an impractical number of SKUs, thus providing sought after benefits in a cost-efficient and storage efficient manner.

### Example Methods

The following discussion now refers to a number of methods and method acts that may be performed. Although the method acts may be discussed in a certain order or illustrated in a flow chart as occurring in a particular order, no particular ordering is required unless specifically stated, or required because an act is dependent on another act being completed prior to the act being performed.

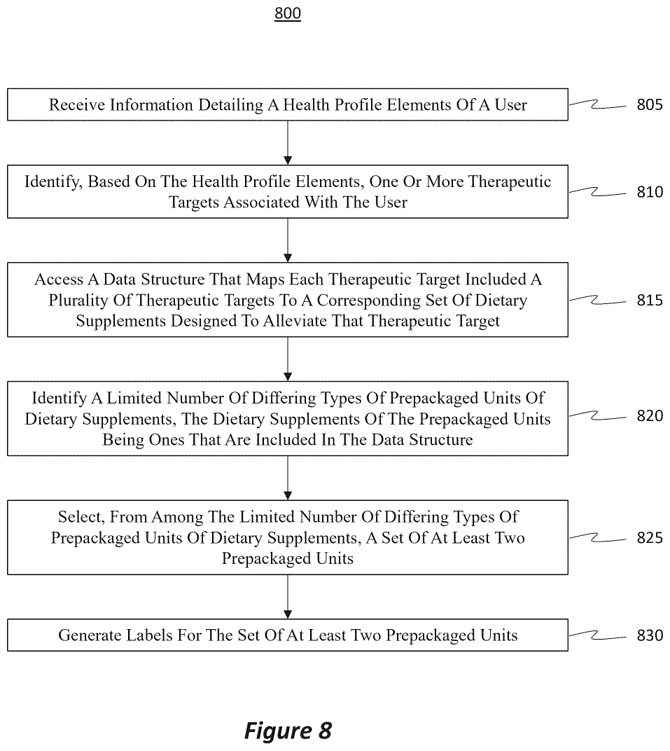

Attention will now be directed to FIG. 8, which illustrates an example method 800 for personalizing sets of dietary supplements for use across a plurality of profiles and for personalizing a particular set of dietary supplements for a specific user. Method 800 can be implemented within the architectures 100A and 100B of FIGS. 1A and 1B and can be performed by the services 105A and 105B.

Method 800 includes an act (act 805) of receiving information detailing health profile elements of a user. In some cases, the information detailing the user's health profile elements can be obtained in part from the user (e.g., such as based on answers to a survey) and in part from one or more additional sources over a network (e.g., information obtained from a medical professional's office). For instance, FIG. 1A shows how the service 105A can acquire the user's health profile elements 115B. The service 105A can also acquire the medical information 140. The health profile elements can include specific health problems the user is currently facing, risk factors the user may potentially face, and/or health-related goals.

The embodiments enable the user to manage what is included in his/her profile. That is, a user profile can be generated for the user. The user profile can be configured to include the user's health profile elements, health history, health goals, or any other information. The user can have control over this profile.

Act 810 includes identifying, based on the health profile elements, one or more therapeutic conditions that may be associated with the user (identified as therapeutic targets). Notably, these therapeutic targets may not necessarily be ones that the user is currently facing, though in some scenarios that may be the case. Thus, the term “associated,” when used in this context, should not be viewed as a requirement that the user actually have a particular therapeutic condition.

As an example, suppose the user's health profile elements indicated a set of risk factors that the user may face in the future, such as perhaps based on the user's family history. Although the user is not currently facing a particular therapeutic condition, the embodiments can infer or deduce that the user might potentially face the therapeutic condition in the future based on the user's risk factors and therefore identify a corresponding therapeutic target for the user. Thus, this therapeutic condition can be identified as a target based on the information in the user's health profile elements.

As another example, suppose the user's health profile elements indicated a set of health-related goals the user would like to focus on or improve. Based on these goals, the embodiments can identify therapeutic conditions on which to focus in order to provide a recommendation regarding which supplement ingredients the user should take. To illustrate, suppose the user expressed a goal of improving his/her ability to focus at work. Individuals who have ADHD also have trouble focusing on a task. In this situation, the embodiments may select ADHD as a therapeutic condition to target based on the user's stated goals. The embodiments can then select the supplement ingredients used to address ADHD as the supplement ingredients to recommend to the user to help improve his/her ability to focus on work.

Accordingly, any type of health profile element can be acquired. The health profile elements are not limited to diseases. For instance, the user may have infrequent joint pain or perhaps occasional mood issues. The user's profile can be updated to include these identified health profile elements, and corresponding therapeutic conditions can be identified as therapeutic targets for the user.

Act 815 includes accessing a data structure that maps each therapeutic condition included a plurality of therapeutic conditions to a corresponding set of dietary supplement ingredients designed to address each therapeutic condition. For instance, the mapping 130A from FIG. 1A can be an example of this “data structure.” The mapping 130A maps the supplements in the supplement palette 125 to various therapeutic conditions. In some example implementations, a machine learning engine can contribute to the data structure by identifying and/or modifying various mappings.

In some embodiments, the set of dietary supplements are included in a set of SKUs, and the formulas in the SKUs are multi-purposefully over-formulated (e.g., principle #2-multi-purpose over-formulation). As a result, the formulas in the SKUs, which include various dietary supplements, are usable to alleviate multiple therapeutic conditions. As an example, zinc and magnesium can be used to alleviate sleep issues as well as immunity issues. The embodiments can capitalize on the recognition (as documented in the mapping 130A) that a single supplement ingredient can be used to alleviate multiple therapeutic conditions.

In some implementations, a machine learning engine is involved in generating and/or updating the data structure. For instance, as additional research emerges expanding on the use of supplements, the mapping 130A from FIG. 1A can be expanded to include this new information as well. In some implementations, the machine learning engine can use user feedback to generate and/or update the data structure. For instance, if a threshold number of users respond by indicating that a particular blend of supplements can be improved by modifying their potencies/dosages, then the ML engine can update the mapping 130A.

Returning to FIG. 8, act 820 includes identifying a limited number of differing types of prepackaged units (i.e., SKUs) of dietary supplement ingredients. The dietary supplement ingredients of the prepackaged units are ones that are included in the data structure. The phrase “limited number of differing types of prepackaged units of dietary supplement ingredients” refers to the notion that different combinations of SKUs can be used to provide different formulas or blends of supplement ingredients to a user, where those formulas are distributed across the different SKUs in a combinatorial manner (combinatorial daily dosing) and where those formulas are optionally multi-purposefully over-formulated. In some cases, the ML engine can submit one or more supplement recommendations (e.g., a recommended daily dose) to the user based on the user's profile.

The embodiments achieve high levels of personalization without requiring vast, impractical numbers of blends and associated SKUs. As one example, FIG. 4 shows a limited number of differing types of prepackaged units. In this case, there are 8 total bottles such that there are 8 differing types of prepackaged units. The embodiments are able to limit the number of differing types of prepackaged units while still achieving high levels of personalization, as described throughout this document. By doing so, the warehousing issue is solved because only a limited number of different types of units need be stored to achieve vast levels of personalization. The issues associated with storing impractically large numbers of SKUs in the warehouse is thus essentially eliminated.

In some embodiments, the number of different types of prepackaged units is between 2 and 80 (e.g., perhaps 40 morning bottle options and 40 evening bottle options). The use of 80 different SKU or bottle types allows for up to about 1,600 different combinations. Thus, initially, at least 1,600 different profiles can be serviced. Of course, the principles described herein may be implemented using more than 80 different types of prepackaged units. By incorporating the notion of multi-purpose over-formulation and dynamic personalization, the number of unique profiles can be further increased, thereby allowing for an almost unlimited number of unique profiles to be serviced. In one example scenario, the limited number of differing types of prepackaged units of dietary supplements includes at least four differing types of prepackaged units designated for morning consumption. The limited number further includes at least four differing types of prepackaged units designated for evening consumption.

Returning to FIG. 8, act 825 includes selecting, from among the limited number of differing types of prepackaged units of dietary supplements, a set of at least two prepackaged units. The set of at least two prepackaged units includes dietary supplement ingredients designed to address the user's one or more therapeutic targets, with the daily dose divided over the at least two prepackaged units (e.g., principle #1—combinatorial daily dosing). In some cases, a machine learning engine can be involved in this selection process to select the set of at least two prepackaged units. The set of units can be configured to include a unit designated for morning consumption by the user and a unit designated for evening consumption by the user. In some cases, the selected number of units is more than 2, such as perhaps 3 or 4. Increasing the number of daily dosing units further increases the number of profiles that can be supported, thereby further increasing the number of potential combinations of prepackaged units to form the recommended daily dose of supplements ingredients, and thereby further increasing the level of personalization.

Act 830 includes generating labels for the set of at least two prepackaged units. The labels are designed to identify correlations between the dietary supplement ingredients included in the set of at least two prepackaged units and the one or more therapeutic targets of the user. Therefore, despite the dietary supplement ingredients included in the set of at least two prepackaged units being usable to alleviate multiple different therapeutic conditions, the labels are designed to emphasize the correlations that are relevant to the one or more therapeutic targets of that specific user (e.g., principle #3—dynamic personalization).

In some cases, the user's profile can be updated to further include an indication reflective of the set of at least two prepackaged units (e.g., a historical record or log detailing what supplements the user has previously used). Additionally, the embodiments enable the user to provide feedback. The user's feedback can also be included in the user's profile. In some cases, the feedback can be used to modify supplement blends and/or potencies/dosages. In some cases, the feedback can be used to modify which sets of prepackaged units the user is provided. In some implementations, feedback is received, where the feedback ranks a utility (e.g., success rate or efficacy) of the set of at least two prepackaged units of dietary supplements in addressing the user's one or more therapeutic targets. In some cases, the feedback is consumed by a machine learning engine, and the machine learning engine modifies the mapping data structure based on the feedback.

### Packets of Supplements

Some embodiments can optionally package the supplement ingredients in the form of a packet or envelope. In some cases, each multivitamin can be a proprietary blend of ingredients and potencies. If 100 proprietary blends of multivitamins are available in stock, the embodiments can provide supplement ingredients for essentially an unlimited number of different profiles. For instance, different combinations of supplements can be generated, thereby servicing any number of profiles.

As an example, suppose every multivitamin has about 20 different ingredients. Embodiments are able to combine different multivitamins to achieve different combinations, where those combinations can be used to service any number of different profiles. The packets/pouch approach can also capitalize on one or more of the combinatorial daily dosing, multi-purpose over-formulation, and dynamic personalization techniques mentioned herein.

In some cases, multiple benefits can be provided with a packet/pouch approach. For instance, the embodiments can allow for the consumption of multiple pills obtained from a packet (where each pill includes multiple supplement ingredients) rather than the consumption of a single pill obtained from a bottle (where each pill also includes multiple supplement ingredients). By allowing for the consumption of multiple pills (and/or other dosage forms), the embodiments can increase the number of supplement ingredients that can be provided to the user. The embodiments can also divide the daily dose between two (or more) pouches/packets according to the combinatorial daily dosing principle discussed herein. Doing so significantly increases both the number of different supplement ingredients that can be provided to the user and the number of unique user profiles that can be serviced.

### Example Computer/Computer Systems

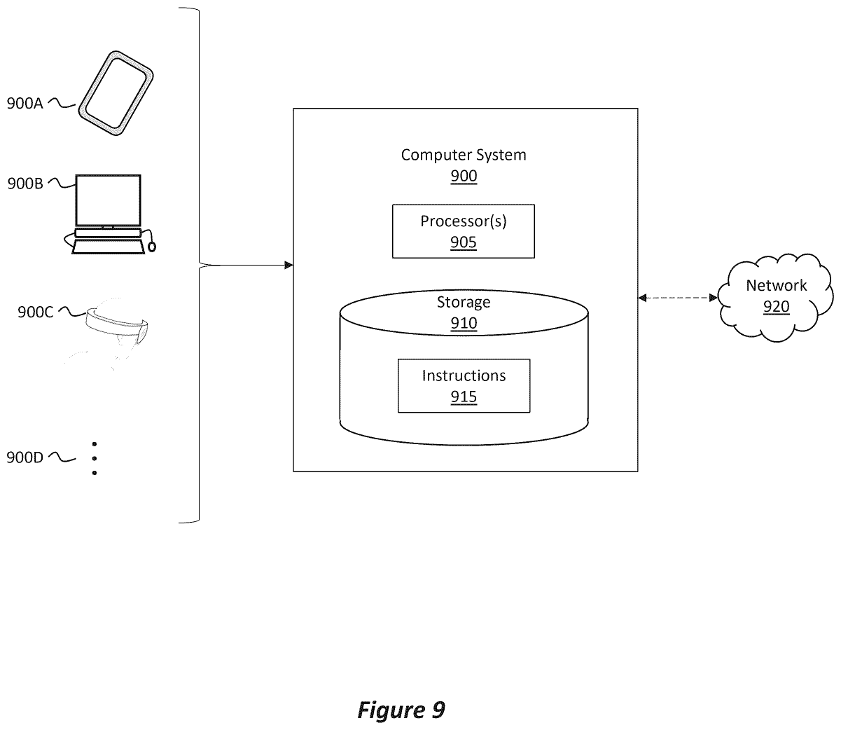

Attention will now be directed to FIG. 9 which illustrates an example computer system 900 that may include and/or be used to perform any of the operations described herein. For instance, computer system 900 can implement the service 105 of FIG. 1 and/or the ML engine 110. Computer system 900 may take various different forms. For example, computer system 900 may be embodied as a tablet 900A, a desktop or a laptop 900B, a wearable device 900C, a mobile device, or any other standalone device, as represented by the ellipsis 900D. Computer system 900 may also be a distributed system that includes one or more connected computing components/devices that are in communication with computer system 900.

In its most basic configuration, computer system 900 includes various different components. FIG. 9 shows that computer system 900 includes one or more processor(s) 905 (aka a “hardware processing unit”) and storage 910.

Regarding the processor(s) 905, it will be appreciated that the functionality described herein can be performed, at least in part, by one or more hardware logic components (e.g., the processor(s) 905). For example, and without limitation, illustrative types of hardware logic components/processors that can be used include Field-Programmable Gate Arrays (“FPGA”), Program-Specific or Application-Specific Integrated Circuits (“ASIC”), Program-Specific Standard Products (“ASSP”), System-On-A-Chip Systems (“SOC”), Complex Programmable Logic Devices (“CPLD”), Central Processing Units (“CPU”), Graphical Processing Units (“GPU”), or any other type of programmable hardware.

As used herein, the terms “executable module,” “executable component,” “component,” “module,” or “engine” can refer to hardware processing units or to software objects, routines, or methods that may be executed on computer system 900. The different components, modules, engines, and services described herein may be implemented as objects or processors that execute on computer system 900 (e.g. as separate threads).

Storage 910 may be physical system memory, which may be volatile, non-volatile, or some combination of the two. The term “memory” may also be used herein to refer to non-volatile mass storage such as physical storage media. If computer system 900 is distributed, the processing, memory, and/or storage capability may be distributed as well.

Storage 910 is shown as including executable instructions 915. The executable instructions 915 represent instructions that are executable by the processor(s) 905 of computer system 900 to perform the disclosed operations, such as those described in the various methods.

The disclosed embodiments may comprise or utilize a special-purpose or general-purpose computer including computer hardware, such as, for example, one or more processors (such as processor(s) 905) and system memory (such as storage 910), as discussed in greater detail below. Embodiments also include physical and other computer-readable media for carrying or storing computer-executable instructions and/or data structures. Such computer-readable media can be any available media that can be accessed by a general-purpose or special-purpose computer system. Computer-readable media that store computer-executable instructions in the form of data are “physical computer storage media” or a “hardware storage device.” Furthermore, computer-readable storage media, which includes physical computer storage media and hardware storage devices, exclude signals, carrier waves, and propagating signals. On the other hand, computer-readable media that carry computer-executable instructions are “transmission media” and include signals, carrier waves, and propagating signals. Thus, by way of example and not limitation, the current embodiments can comprise at least two distinctly different kinds of computer-readable media: computer storage media and transmission media.

Computer storage media (aka “hardware storage device”) are computer-readable hardware storage devices, such as RAM, ROM, EEPROM, CD-ROM, solid state drives (“SSD”) that are based on RAM, Flash memory, phase-change memory (“PCM”), or other types of memory, or other optical disk storage, magnetic disk storage or other magnetic storage devices, or any other medium that can be used to store desired program code means in the form of computer-executable instructions, data, or data structures and that can be accessed by a general-purpose or special-purpose computer.

Computer system 900 may also be connected (via a wired or wireless connection) to external sensors (e.g., one or more remote cameras) or devices via a network 920. For example, computer system 900 can communicate with any number devices or cloud services to obtain or process data. In some cases, network 920 may itself be a cloud network. Furthermore, computer system 900 may also be connected through one or more wired or wireless networks to remote/separate computer systems(s) that are configured to perform any of the processing described with regard to computer system 900.

A “network,” like network 920, is defined as one or more data links and/or data switches that enable the transport of electronic data between computer systems, modules, and/or other electronic devices. When information is transferred, or provided, over a network (either hardwired, wireless, or a combination of hardwired and wireless) to a computer, the computer properly views the connection as a transmission medium. Computer system 900 will include one or more communication channels that are used to communicate with the network 920. Transmissions media include a network that can be used to carry data or desired program code means in the form of computer-executable instructions or in the form of data structures. Further, these computer-executable instructions can be accessed by a general-purpose or special-purpose computer. Combinations of the above should also be included within the scope of computer-readable media.

Upon reaching various computer system components, program code means in the form of computer-executable instructions or data structures can be transferred automatically from transmission media to computer storage media (or vice versa). For example, computer-executable instructions or data structures received over a network or data link can be buffered in RAM within a network interface module (e.g., a network interface card or “NIC”) and then eventually transferred to computer system RAM and/or to less volatile computer storage media at a computer system. Thus, it should be understood that computer storage media can be included in computer system components that also (or even primarily) utilize transmission media.

Computer-executable (or computer-interpretable) instructions comprise, for example, instructions that cause a general-purpose computer, special-purpose computer, or special-purpose processing device to perform a certain function or group of functions. The computer-executable instructions may be, for example, binaries, intermediate format instructions such as assembly language, or even source code. Although the subject matter has been described in language specific to structural features and/or methodological acts, it is to be understood that the subject matter defined in the appended claims is not necessarily limited to the described features or acts described above. Rather, the described features and acts are disclosed as example forms of implementing the claims.

Those skilled in the art will appreciate that the embodiments may be practiced in network computing environments with many types of computer system configurations, including personal computers, desktop computers, laptop computers, message processors, hand-held devices, multi-processor systems, microprocessor-based or programmable consumer electronics, network PCs, minicomputers, mainframe computers, mobile telephones, PDAs, pagers, routers, switches, and the like. The embodiments may also be practiced in distributed system environments where local and remote computer systems that are linked (either by hardwired data links, wireless data links, or by a combination of hardwired and wireless data links) through a network each perform tasks (e.g. cloud computing, cloud services and the like). In a distributed system environment, program modules may be located in both local and remote memory storage devices.

### Example Health Profile and Supplement Recommendations

The following example illustrates one potential health profile and an example formula (i.e., set of supplement ingredients) recommended to the user according to an embodiment of the presently disclosed systems and methods. It will be understood that this example is for purposes of illustration only and is not intended to be limiting. For example, other embodiments may include a different set of health profile elements and/or different set of recommended supplement ingredients. Moreover, other users will likely have different responses to a health profile survey.

The table below provides an example set of health profile elements that collectively form at least a portion of an example user's health profile. As shown, the health profile elements may be obtained from a variety of sources such as a health survey, clinical data (e.g., blood testing), and genetic testing data. Other examples may omit one or more of such sources, and not all of the example sources are required to implement the disclosed systems and methods. The example also illustrates that health profile elements may include one or more of current health problems/conditions, risk factors, and health goals.

| | |
|---|---|
| Biological Data (7 variables): | Basic Genetics Data (7 variables): |
| Gender = Male | A doctor or gene testing service indicated I |
| Birthday = Jun. 4, 1963 | have one of the following mutations: |
| Age = 58 | Heart (like a disposition to heart disease) = Yes |
| Height = 6′4″ | Mood/Mental Health (like a disposition |
| Weight = 210 | toward depression) = Yes |
| BMI = 26.77 | Brain Conditions (including risk of |
| BMI Category = Slightly Overweight | Alzheimer's) = Yes |
| | Methylation Cycle (inability to absorb |
| | certain non-methylated nutrients) = Yes |
| | Weight Control = Yes |
| | Have you been told you have the MTHFR |
| | Gene mutation = Yes |
| | Do you have access to detailed Genetic |
| | Testing Results = Yes |
| Top 15 Health Priorities Data (15 variables): | Diet Data (13 variables): |
| Heart Disease | Diet Description = Vegetarian |
| High Cholesterol | Lactose Intolerant = No |
| Exercise | Gluten Intolerant = No |
| Brain Fog | Celiac Disease = No |
| Cognition Problems | Fruit Consumption = Not Enough |
| Alzheimer's | Vegetable Consumption = Not Enough |
| Diabetes | Fiber Consumption = Just Right |
| High Blood Pressure | Protein Consumption = Not Enough |
| Anxiety | Egg Consumption = Just Right |
| Depression | Sugar Consumption = Too Much |
| Gas/Bloating | Red Meat Consumption = Less than Once a Week |
| Eye Problems | Salmon Consumption = Less than Once a Week |
| Sleep Issues | Other Fish Consumption = Less than Once a Week |
| Hair Problems | Other Meat Consumption = Less than Once a Week |
| Allergies | |
| Health Goals Data (21 variables): | Vitamin Deficiency Data (20 variables): |
| Great Heart Health = Yes | Blood tests have indicated deficiencies with: |
| Better Sleep = Yes | Vitamin A = No |
| Athletic/Exercise Performance = Yes | Vitamin C = No |
| Preserve Eyesight = Yes | Vitamin D = Yes |
| Beautiful Hair, Skin, Nails = Yes | Vitamin E = No |
| Stronger Immunity = Yes | Vitamin K = No |
| Headache Reduction = No | Vitamin B1 Thiamine = No |
| Joint Pain Reduction = Yes | Vitamin B2 Riboflavin = No |
| Better Brain = Yes | Vitamin B3 Niacin = No |
| Calmness = Yes | Vitamin B5 Pantothenic Acid = No |
| Stress/Anger Management = Yes | Vitamin B6 Pyridoxine = No |
| Happier Mood = Yes | Vitamin B7 Biotin = No |
| Detoxification = Yes | Vitamin B9 Folate = Yes |
| Weight Control = Yes | Vitamin B12 Cobalamin = Yes |
| Sexual Health = Yes | Calcium = No |
| Improved Fertility = No | Iron = No |
| Good Digestion = Yes | Magnesium = No |
| General Wellness = Yes | Manganese = No |
| Anti-Aging = Yes | Potassium = No |
| Dental Health = No | Zinc = No |
| Blood Sugar Control = Yes | Omega 3 = No |
| Lifestyle Data (25 variables): | Medical Background Data (20 variables): |
| Zip Code I live in = 84018 | Excluding Dietary Allergies = Shellfish |
| Sun Exposure Location = Low Sun Zone | Ignore Excluding Allergies = Yes |
| Stress level for life right now = Very High | Environmental Allergies = Ragweed, |
| I live in a major city or industrial area = Yes | Grass, Pollen |
| I work night or swing shifts = No | \hat{ }Female Health Status = NA (male) |
| I have a long or taxing commute = No | \hat{ }Menopause Adaptation Status = NA (male) |
| I am regularly exposed to unrelenting | I struggle with low energy = No |
| chaotic or noise = No | I wear glasses or contacts = Yes |
| I am regularly exposed to a lot of sick | I have had a recent dramatic change in eyesight = No |
| people = No | I am currently taking prescription |
| I am able to sleep in a dark, quiet room = Yes | antidepressants = Yes |
| I regularly wear sunscreen = Yes | I have had some heart related surgery = No |
| I exercise (30 minutes+) _{—}_{——} times a week = 5 | I frequently get sick with colds = No |
| Exercise Category = Good Exercise Levels | I frequently get infections = No |
| I use stimulants (like coffee or energy | When I get sick, I struggle to get over it = No |
| drink) to wake up = No | I have used antibiotics five or more times = Yes |
| I am often using stimulants near bedtime = No | I have recently used antibiotics = No |
| I use wine/alcohol to calm down in | Abdominal Fat = Moderate |
| evening = No | I use sleeping pills to get to sleep = Never |
| I use psychoactive drugs to manage mood = No | Do you have headaches = Rarely |
| I typically sleep _{—}_{——} hours a night = 9 | Sunburns in my life = Very Many |
| Sleep Category = Healthy Sleep Levels | Brain Concussions in life = 1-2 |
| I typically meditate _{—}_{——} days a week = 7 | |
| Meditation Category = High Meditation | |
| Alcohol Consumption = None | |
| Tobacco Consumption = None | |
| Caffeine Consumption = None | |
| Exposure to Air Pollution = Moderate | |
| Exposure to Environmental Toxins = Moderate | |
| Current Problems Data (60 variables): | Family History Data (60 variables): |
| High Cholesterol = Yes | High Cholesterol = Yes |
| Heart Disease = Yes | Heart Disease = Yes |
| High Blood Pressure = Yes | High Blood Pressure = Yes |
| Atherosclerosis = No | Atherosclerosis = Yes |
| High Homocysteine = Yes | High Homocysteine = Yes |
| Blood Flow, Circulation Problems = No | Blood Flow, Circulation Problems = Yes |
| Brain Fog = Yes | Brain Fog = Yes |
| Cognition Problems = Yes | Cognition Problems = Yes |
| Memory Problems = Yes | Memory Problems = Yes |
| Alzheimer's/Dementia = No | Alzheimer's/Dementia = Yes |
| Depression = Yes | Depression = Yes |
| Anxiety = Yes | Anxiety = Yes |
| Problems with Calmness/Relaxation = Yes | Problems with Calmness/Relaxation = Yes |
| Problems with Feeling Subjective | Problems with Feeling Subjective |
| Wellbeing = Yes | Wellbeing = Yes |
| Seasonal Affective Disorder = No | Seasonal Affective Disorder = No |
| Problems with Anger/Aggression = No | Problems with Anger/Aggression = Yes |
| Bi-Polar = No | Bi-Polar = No |
| Stress Issues = Yes | Stress Issues = Yes |
| Fatigue = No | Fatigue = Yes |
| Respiratory Problems = No | Respiratory Problems = Yes |
| Acne = No | Acne = Yes |
| Non-Acne Skin Problems = Yes | Non-Acne Skin Problems = Yes |
| Chapped Lips = No | Chapped Lips = Yes |
| Hair Problems = Yes | Hair Problems = Yes |
| Nail Problems = No | Nail Problems = No |
| Weight Problems = Yes | Weight Problems = Yes |
| Appetite Control Problems = Yes | Appetite Control Problems = Yes |
| Sleep Issues = No | Sleep Issues = Yes |
| Athletic Performance Problems = No | Athletic Performance Problems = Yes |
| Exercise/Training Problems = No | Exercise/Training Problems = Yes |
| Joint Pain = Yes | Joint Pain = Yes |
| Diabetes = Yes | Diabetes = Yes |
| Glycemic Control Problems = Yes | Glycemic Control Problems = Yes |
| Cancer = No | Cancer = Yes |
| Abdominal Discomfort = No | Abdominal Discomfort = Yes |
| Problems Digesting Foods = No | Problems Digesting Foods = Yes |
| Gas/Bloating = Yes | Gas/Bloating = Yes |
| BM Irregularity = No | BM Irregularity = Yes |
| Poor Gut Health = No | Poor Gut Health = Yes |
| Eye Problems = Yes | Eye Problems = Yes |
| Inflammation = Yes | Inflammation = Yes |
| Erectile Disfunction = No | Erectile Disfunction = No |
| Low Testosterone = Yes | Low Testosterone = Yes |
| Male/Sperm Infertility = No | Male/Sperm Infertility = No |
| Male Libido Problems = No | Male Libido Problems = No |
| Prostate Issues = Yes | Prostate Issues = Yes |
| \hat{ }Vaginal Health Problems = NA (male) | \hat{ }Vaginal Health Problems = NA (male) |
| \hat{ }Female Infertility = NA (male) | \hat{ }Female Infertility = NA (male) |
| \hat{ }Female Libido Problems = NA (male) | \hat{ }Female Libido Problems = NA (male) |
| \hat{ }Menstruation/PMS Issues = NA (male) | \hat{ }Menstruation/PMS Issues = NA (male) |
| \hat{ }Menopause = NA (male) | \hat{ }Menopause = NA (male) |
| Migraines/Headaches = No | Migraines/Headaches = Yes |
| Allergies = Yes | Allergies = Yes |
| Asthma = No | Asthma = Yes |
| Liver Kidney Issues/Toxins = No | Liver Kidney Issues/Toxins = Yes |
| Metabolic or Cellular Issues = No | Metabolic or Cellular Issues = Yes |
| Dental Health = No | Dental Health = Yes |
| Osteoporosis = No | Osteoporosis = No |
| Strokes = No | Strokes = No |
| Premature Aging = No | Premature Aging = No |
| Past Problems Data (60 variables): | Detailed Genetics Data (27 variables): |
| High Cholesterol = Yes | I have a one or two allele gene mutation on |
| Heart Disease = Yes | the following genes: |
| High Blood Pressure = Yes | MTHFR 677 = Yes |
| Atherosclerosis = No | MTHFR 1298 = Yes |
| High Homocysteine = Yes | MTRR = Yes |
| Blood Flow, Circulation Problems = No | MAT1 = No |
| Brain Fog = Yes | COMT = No |
| Cognition Problems = Yes | PEMT = No |
| Memory Problems = Yes | CYPWRI = No |
| Alzheimer's/Dementia = No | CBS = No |
| Depression = Yes | BCMO1 = No |
| Anxiety = Yes | FADS2 = No |
| Problems with Calmness/Relaxation = Yes | FUT2 = No |
| Problems with Feeling Subjective | NBPF3 = No |
| Wellbeing = Yes | ADIPOQ = No |
| Seasonal Affective Disorder = No | HNMT = No |
| Problems with Anger/Aggression = Yes | GAD1 = No |
| Bi-Polar = No | FAAH = No |
| Stress Issues = Yes | APOE = Yes |
| Fatigue = Yes | SOD2 = No |
| Respiratory Problems = Yes | SOD3 = No |
| Acne = Yes | NOS1 = No |
| Non-Acne Skin Problems = Yes | CFH = No |
| Chapped Lips = Yes | TP53 = No |
| Hair Problems = Yes | GATA3 = No |
| Nail Problems = Yes | PON1 = No |
| Weight Problems = Yes | 9p21 = No |
| Appetite Control Problems = Yes | FADS1 = No |
| Sleep Issues = Yes | ADD1 = No |
| Athletic Performance Problems = Yes | Hormone Deficiency Data (3 variables): |
| Exercise/Training Problems = Yes | Blood tests have indicated deficiencies with: |
| Joint Pain = Yes | Testosterone = Yes |
| Diabetes = Yes | Estrogen = No |
| Glycemic Control Problems = Yes | Progesterone = No |
| Cancer = No | |
| Abdominal Discomfort = Yes | |
| Problems Digesting Foods = Yes | |
| Gas/Bloating = Yes | |
| BM Irregularity = No | |
| Poor Gut Health = Yes | |
| Eye Problems = Yes | |
| Inflammation = Yes | |
| Erectile Disfunction = Yes | |
| Low Testosterone = Yes | |
| Male/Sperm Infertility = No | |
| Male Libido Problems = Yes | |
| Prostate Issues = Yes | |
| \hat{ }Vaginal Health Problems = NA (male) | |
| \hat{ }Female Infertility = NA (male) | |
| \hat{ }Female Libido Problems = NA (male) | |
| \hat{ }Menstruation/PMS Issues = NA (male) | |
| \hat{ }Menopause = NA (male) | |
| Migraines/Headaches = Yes | |
| Allergies = Yes | |
| Asthma = Yes | |
| Liver Kidney Issues/Toxins = Yes | |
| Metabolic or Cellular Issues = No | |
| Dental Health = Yes | |
| Osteoporosis = No | |
| Strokes = No | |
| Premature Aging = No | |
| Basic Blood Data (8 variables): | Detailed Blood Test Data (88 variables): |
| Has a doctor conducted blood tests on you | Actual Value of Test/Reference Interval/ |
| that indicated that you have any of the | Date of Test |
| following issues: | CMP - Glucose 103 mg/dL/[HIGH]/ |
| High Cholesterol = Yes | Jul. 21, 2021 |
| High Homocysteine = Yes | CMP - Calcium 10.5 mg/dL/[HIGH]/ |
| Diabetes = No | Jul. 21, 2021 |
| Pre-Diabetes = Yes | CMP - Sodium 147 mmol/L/[HIGH]/ |
| Any Vitamin Deficiency = Yes | Jul. 21, 2021 |
| Any Hormone Deficiency = Yes | CMP - Potassium 5.1 mg/dL/ |
| Access to detailed Blood Testing Results = Yes | [NORMAL]/Jul. 21, 2021 |
| | CMP - Bicarbonate (CO2) _{———}? |
| | CMP - Chloride 106 mmol/L/ |
| | [NORMAL]/Jul. 21, 2021 |
| | CMP - BUN (Blood Urea Nitrogen) 18 |
| | mg/dL/[NORMAL]/Jul. 21, 2021 |
| | CMP - Creatinine 1.12 mg/dL/ |
| | [NORMAL]/Jul. 21, 2021 |
| | CMP - BUN/Creatinine Ratio 16% |
| | Jul. 21, 2021 |

As disclosed herein, the system can identify one or more therapeutic targets based on the example user's health profile elements. These therapeutic targets are mapped to a set of one or more dietary supplement ingredients. The following represents and example set of dietary supplement ingredients for this example user based on the above health profile elements. The recommended daily dosages of these supplement ingredients are beneficially divided into separate prepackaged units that are multi-purposefully over-formulated (as those terms are described above).

### Morning Formula Example (Serving Size 4 Pills):

Basic Blend (1501 mg): Acetyl L-Carnitine, Zinc, L-Carnitine (non-Acetylated form), Cocoa Extract, Ginger, Creatine, B9 Folate, B12 Cobalamin, Resveratrol, Chromium, B6 Pyridoxine, B3 Niacin, DHEA, Vitamin C, N-Acetylcysteine (NAC), L-Tyrosine, 5-HTP, Beetroot, B7 Biotin, B2 Riboflavin, B1 Thiamine, Boron, Manganese, B5 Pantothenic Acid, Apple Cider Vinegar. Athlete Blend (260 mg): Leucine (BCAA), Beta-Alanine.
Brain Blend (150 mg): Lion's Mane, Bacopa.
Digestion Blend (268 mg): Peppermint Oil, Olive leaf extract, Colostrum, Chlorogenic Acid, Chaga.
Energy Blend (241 mg): Pyrroloquinoline quinone (PQQ), Iron, Green Tea Catechins, Coleus forskohlii.
Immunity Blend (235 mg): Holy Basil, Cranberry, Berberine, Banaba, Astragalus.
Probiotics Blend (295 mg): Saccharomyces boulardii, Lactobacillus acidophilus, Lactobacillus (Limosilactobacillus) reuteri, Lactobacillus (Lactiplantibacillus) plantarum, Lactobacillus (Lacticaseibacillus) casei, Inulin, Bifidobacterium longum, Bifidobacterium lactis, Bifidobacterium infantis, Bifidobacterium breve, Bifidobacterium bifidum.
Supergreens Blend (750 mg): Spirulina, Red Cabbage, Moringa, Chlorella, Carrot.

### Evening Formula Example (Serving Size 4 Pills):

Basic Blend (2393 mg): Panax Ginseng, DHA, Saffron, Gingko Biloba, Vitamin D3, Curcumin, Thymoquinone, Ashwagandha, CoQ10, Rhodiola Rosea, Gotu Kola, Vitamin E, Lemon Balm, Inositol, EPA, Vitamin A, Garlic, Reishi, Maca Root, Cissus quadrangularis, Pelargonium sidoides, MSM, Glucosamine, Calcium, Oil of Oregano, Vitamin K2, Magnesium (General). Male/Female Blend 1 (530 mg): Royal Jelly, Maritime Pine Bark Extract, Astaxanthin, Vitex Berry, Collagen, Magnolia officinalis, Sage, Oleoylethanolamide, Black Cohosh.
Male/Female Blend 2 (509 mg): Tongkat ali, Tribulus, Fenugreek, Stinging Nettle, Saw Palmetto, Pygeum, Pumpkin Seed, Beta-Sitosterol.
Sleep/Aging Blend (268 mg): Melatonin, Theanine, Kava, Valerian Root, Lutein.

### Example Aspects

In view of the foregoing, the present invention relates, for example and without being limited thereto, to the following aspects:

In a first aspect, a method for personalizing sets of dietary supplements for use across a plurality of profiles and for personalizing a particular set of dietary supplements for a specific user, said method comprising: receiving information detailing a health profile element of a user; identifying, based on the health profile element, one or more therapeutic conditions associated with the user (e.g., and defining such as therapeutic targets of the user); accessing a data structure that maps each therapeutic condition included in a plurality of therapeutic conditions to a corresponding set of dietary supplements designed to alleviate each therapeutic condition, wherein: different formulas of dietary supplements are included in different sets of prepackaged units (e.g., SKUs) that are multi-purposefully over-formulated such that said formulas in the different sets of prepackaged units are usable to alleviate multiple therapeutic conditions; identifying a limited number of differing types of the prepackaged units of dietary supplements, the dietary supplements of the prepackaged units being ones that are included in the data structure; selecting, from among the limited number of differing types of prepackaged units of dietary supplements, a set of at least two prepackaged units, wherein: said selecting is based on the user's health profile element and on the data structure, the set of at least two prepackaged units constitute a divided daily dosage for the user, and the set of at least two prepackaged units includes dietary supplements designed to address the one or more therapeutic conditions associated with the user; and generating labels for the set of at least two prepackaged units, wherein the labels are designed to identify correlations between the dietary supplements included in the set of at least two prepackaged units and the one or more therapeutic targets of the user such that, despite the dietary supplements included in the set of at least two prepackaged units being usable to address multiple different therapeutic conditions, the labels are designed to emphasize the correlations that are relevant to the user's one or more therapeutic targets.

In a second aspect as recited above, wherein a user profile is generated for the user, and wherein the user profile includes the user's health profile element and further includes an indication reflective of the set of at least two prepackaged units.

In a third aspect as recited in any of the preceding aspects, wherein the information detailing the health profile element of the user is obtained in part from the user and in part from one or more additional sources over a network.

In a fourth aspect as recited in any of the preceding aspects, wherein a machine learning engine contributes to the data structure by identifying various mappings.

In a fifth aspect as recited in any of the preceding aspects, wherein the limited number of differing types of prepackaged units of dietary supplements is between 2 and about 80 different types, optionally greater than 80.

In a sixth aspect as recited in any of the preceding aspects, particularly with regard to aspect 5, wherein the limited number is at a minimum of about 8 differing types.

In a seventh aspect as recited in any of the preceding aspects, wherein feedback is received, where the feedback ranks a utility of the set of at least two prepackaged units of dietary supplements in alleviating the one or more therapeutic targets of the user.

In an eighth aspect as recited in any of the preceding aspects, particularly with regard to aspect 7, wherein the feedback is consumed by a machine learning engine, and wherein the machine learning engine modifies the data structure based on the feedback.

In a ninth aspect as recited in any of the preceding aspects, wherein each of the prepackaged units includes at least a same basic profile blend of dietary supplements, and wherein at least some of the prepackaged units further include additional dietary supplements beyond those that are included in the basic profile blend.

In a tenth aspect as recited in any of the preceding aspects, wherein a user profile is generated for the user, and wherein the user profile is manageable by the user.

In an eleventh aspect, a computer system configured to personalize sets of dietary supplements for use across a plurality of profiles and to personalize a particular set of dietary supplements for a specific user, said computer system comprising: one or more processors; and one or more computer-readable hardware storage devices that store instructions that are executable by the one or more processors to cause the computer system to: receive information detailing a health profile element of a user; identify, based on the health profile element, one or more therapeutic conditions associated with the user (and defining such as one or more therapeutic targets of the user); access a data structure that maps each therapeutic condition included a plurality of therapeutic conditions to a corresponding set of dietary supplements designed to alleviate said each therapeutic condition, wherein: different formulas of dietary supplements are included in different sets of prepackaged units that are multi-purposefully over-formulated such that said formulas in the different sets of prepacked units are usable to alleviate multiple therapeutic conditions; identify a limited number of differing types of the prepackaged units of dietary supplements, the dietary supplements of the prepackaged units being ones that are included in the data structure; select, from among the limited number of differing types of prepackaged units of dietary supplements, a set of at least two prepackaged units, wherein: said selecting is based on the user's health profile elements and on the data structure, the set of at least two prepackaged units constitute a divided daily dosage for the user, and the set of at least two prepackaged units includes dietary supplements designed to alleviate the one or more therapeutic conditions associated with the user; and generate labels for the set of at least two prepackaged units, wherein the labels are designed to identify correlations between the dietary supplements included in the set of at least two prepackaged units and the one or more therapeutic targets of the user such that, despite the dietary supplements included in the set of at least two prepackaged units being usable to alleviate multiple different therapeutic conditions, the labels are designed to emphasize the correlations that are relevant to the one or more therapeutic targets of the user.

In a twelfth aspect as recited in any of the preceding aspects, wherein a number of unique combinations of the differing types of prepackaged units of dietary supplements is at least 1,600.

In a thirteenth aspect as recited in any of the preceding aspects, wherein the set of at least two prepackaged units includes a unit designated for morning consumption by the user and a unit designated for evening consumption by the user.

In a fourteenth aspect as recited in any of the preceding aspects, wherein the limited number of differing types of prepackaged units of dietary supplements includes at least four differing types of prepackaged units designated for morning consumption and at least four differing types of prepackaged units designated for evening consumption.

In a fifteenth aspect as recited in any of the preceding aspects, wherein: the set of at least two prepackaged units of dietary supplements includes a first unit and a second unit, a second user is identified, where the second user has a second set of therapeutic conditions, the first unit is usable to alleviate the second set of therapeutic conditions, and a label generated for the first unit for the user is different as compared to a second label that is generated for the first unit for the second user despite a set of dietary supplements in the first unit not changing.

In a sixteenth aspect as recited in any of the preceding aspects, wherein a machine learning engine is involved in said selecting to select the set of at least two prepackaged units.

In a seventeenth aspect, a computer system configured to personalize sets of dietary supplements for use across a plurality of profiles and to personalize a particular set of dietary supplements for a specific user, said computer system comprising: one or more processors; and one or more computer-readable hardware storage devices that store instructions that are executable by the one or more processors to cause the computer system to: receive information detailing a health profile element of a user; identify, based on the health profile element, one or more therapeutic conditions associated with the user (i.e., a therapeutic target of the user); access a data structure that maps each therapeutic condition included a plurality of therapeutic conditions to a corresponding set of dietary supplements designed to alleviate said each therapeutic condition, wherein: different formulas of dietary supplements are included in different sets of prepackaged units that are multi-purposefully over-formulated such that said formulas in the different sets of prepackaged units are usable to alleviate multiple therapeutic conditions, a machine learning engine is involved in generating and/or updating the data structure, and the machine learning engine uses user feedback to generate and/or update the data structure; identify a limited number of differing types of the prepackaged units of dietary supplements, the dietary supplements of the prepackaged units being ones that are included in the data structure; select, from among the limited number of differing types of prepackaged units of dietary supplements, a set of at least two prepackaged units, wherein: said selecting is based on the health profile element and on the data structure, the set of at least two prepackaged units constitute a divided daily dosage for the user, and the set of at least two prepackaged units includes dietary supplements designed to alleviate the one or more therapeutic targets of the user; and generate labels for the set of at least two prepackaged units, wherein the labels are designed to identify correlations between the dietary supplements included in the set of at least two prepackaged units and the one or more therapeutic targets of the user such that, despite the dietary supplements included in the set of at least two prepackaged units being usable to alleviate multiple different therapeutic conditions, the labels are designed to emphasize the correlations that are relevant to the one or more therapeutic targets of the user.

In an eighteenth aspect as recited in any of the preceding aspects, wherein each dietary supplement in the data structure is tagged based on a content tagging schema.

In a nineteenth aspect as recited in any of the preceding aspects, wherein the set of at least two prepackaged units of dietary supplements includes one or more of a female pack, a male pack, an immunity pack, a brain pack, or a sleep pack.

In a twentieth aspect as recited in any of the preceding aspects, particularly with regard to the nineteenth aspect, wherein the set of at least two prepackaged units of dietary supplements includes one or more of an energy pack, an athlete pack, or a digestion pack.

Any of the aforementioned aspects may be combined with any other of the aforementioned aspects. The present invention may be embodied in other specific forms without departing from its characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A method for personalizing a set of dietary supplements for a specific user, said method comprising:
receiving information detailing a health profile element of a user;
identifying, based on the health profile element, one or more therapeutic conditions associated with the user and defining the one or more therapeutic conditions associated with the user as one or more therapeutic targets of the user;
accessing a data structure that maps each therapeutic condition included a plurality of therapeutic conditions to a corresponding set of dietary supplement ingredients designed to alleviate said each therapeutic condition, wherein:
different formulas of dietary supplement ingredients are included in different sets of prepackaged units that are optionally multi-purposefully over-formulated such that said formulas in the different sets of prepackaged units are usable to alleviate multiple therapeutic conditions;
identifying a limited number of differing types of the prepackaged units of dietary supplements, the dietary supplement ingredients of the prepackaged units being ones that are included in the data structure;
selecting, from among the limited number of differing types of prepackaged units of dietary supplement ingredients, a set of at least two prepackaged units, wherein:
said selecting is based on the user's health profile element and on the data structure,
the set of at least two prepackaged units constitute a divided daily dosage for the user, and
the set of at least two prepackaged units includes dietary supplements designed to alleviate the one or more therapeutic targets of the user; and
generating labels for the set of at least two prepackaged units, wherein the labels are designed to identify correlations between the dietary supplement ingredients included in the set of at least two prepackaged units and the one or more therapeutic targets of the user such that, despite the dietary supplement ingredients included in the set of at least two prepackaged units being usable to alleviate multiple different therapeutic conditions, the labels are designed to emphasize the correlations that are relevant to the one or more therapeutic targets of the user.

2. The method of claim 1, wherein a user profile is generated for the user, and wherein the user profile includes the user's health profile element and further includes an indication reflective of the set of at least two prepackaged units.

3. The method of claim 1, wherein the information detailing the health profile element of the user is obtained in part from the user and in part from one or more additional sources over a network.

4. The method of claim 1, wherein a machine learning engine contributes to the data structure by identifying various mappings.

5. The method of claim 1, wherein the limited number of differing types of prepackaged units of dietary supplement ingredients is between 2 to about 80 differing types.

6. The method of claim 5, wherein the limited number has at least about 4 differing types, or at least about 6 differing types, or at least about 8 differing types.

7. The method of claim 1, wherein feedback is received, where the feedback ranks a utility of the set of at least two prepackaged units of dietary supplement ingredients in alleviating the one or more therapeutic conditions associated with the user.

8. The method of claim 7, wherein the feedback is consumed by a machine learning engine, and wherein the machine learning engine modifies the data structure based on the feedback.

9. The method of claim 1, wherein each of the prepackaged units includes at least a same basic profile blend of dietary supplements, and wherein at least some of the prepackaged units further include additional dietary supplements beyond those that are included in the basic profile blend.

10. The method of claim 1, wherein a user profile is generated for the user, and wherein the user profile is manageable by the user.

11. A computer system configured to personalize a particular set of dietary supplement ingredients for a specific user, said computer system comprising:
one or more processors; and
one or more computer-readable hardware storage devices that store instructions that are executable by the one or more processors to cause the computer system to:
receive information detailing a health profile element of a user;
identify, based on the health profile element, one or more therapeutic conditions associated with the user and defining the one or more therapeutic conditions associated with the user as one or more therapeutic targets of the user;
access a data structure that maps each therapeutic condition included a plurality of therapeutic conditions to a corresponding set of dietary supplement ingredients designed to alleviate said each therapeutic condition, wherein:
different formulas of dietary supplement ingredients are included in different sets of prepackaged units that are optionally multi-purposefully over-formulated such that said formulas in the different sets of prepackaged units are usable to alleviate multiple therapeutic conditions;
identify a limited number of differing types of the prepackaged units of dietary supplement ingredients, the dietary supplement ingredients of the prepackaged units being ones that are included in the data structure;
select, from among the limited number of differing types of prepackaged units of dietary supplement ingredients, a set of at least two prepackaged units, wherein:
said selecting is based on the user's health profile elements and on the data structure,
the set of at least two prepackaged units constitute a divided daily dosage for the user, and
the set of at least two prepackaged units includes dietary supplement ingredients designed to alleviate the one or more therapeutic targets of the user; and
generate labels for the set of at least two prepackaged units, wherein the labels are designed to identify correlations between the dietary supplement ingredients included in the set of at least two prepackaged units and the one or more therapeutic targets of the user such that, despite the dietary supplement ingredients included in the set of at least two prepackaged units being usable to alleviate multiple different therapeutic conditions, the labels are designed to emphasize the correlations that are relevant to the user's one or more therapeutic targets.

12. The computer system of claim 11, wherein a number of unique combinations of the differing types of prepackaged units of dietary supplement ingredients is at least about 20, or at least about 40, or at least about 80, or at least about 120, or at least about 160, or at least about 320, or at least about 640, or at least about 1200, or at least about 1,600.

13. The computer system of claim 11, wherein the set of at least two prepackaged units includes a unit designated for morning consumption by the user and a unit designated for evening consumption by the user.

14. The computer system of claim 11, wherein the limited number of differing types of prepackaged units of dietary supplement ingredients includes at least four differing types of prepackaged units designated for morning consumption and at least four differing types of prepackaged units designated for evening consumption.

15. The computer system of claim 11, wherein:
the set of at least two prepackaged units of dietary supplements includes a first unit and a second unit,
a second user is identified, where the second user has a second set of therapeutic conditions,
the first unit is usable to alleviate the second set of therapeutic conditions, and
a label generated for the first unit for the user is different as compared to a second label that is generated for the first unit for the second user despite a set of dietary supplements in the first unit not changing.

16. The computer system of claim 11, wherein a machine learning engine is involved in said selecting to select the set of at least two prepackaged units.

17. A computer system configured to personalize a particular set of dietary supplements for a specific user, said computer system comprising:
one or more processors; and
one or more computer-readable hardware storage devices that store instructions that are executable by the one or more processors to cause the computer system to:
receive information detailing a health profile element of a user;
identify, based on the health profile element, one or more therapeutic conditions associated with the user and defining the one or more therapeutic conditions associated with the user as one or more therapeutic targets of the user;
access a data structure that maps each therapeutic condition included a plurality of therapeutic conditions to a corresponding set of dietary supplement ingredients designed to alleviate said each therapeutic condition, wherein:
different formulas of dietary supplement ingredients are included in different sets of prepackaged units that are optionally multi-purposefully over-formulated such that said formulas in the different sets of prepackaged units are usable to alleviate multiple therapeutic conditions,
a machine learning engine is involved in generating and/or updating the data structure, and the machine learning engine uses user feedback to generate and/or update the data structure;
identify a limited number of differing types of the prepackaged units of dietary supplement ingredients, the dietary supplements of the prepackaged units being ones that are included in the data structure;
select, from among the limited number of differing types of prepackaged units of dietary supplement ingredients, a set of at least two prepackaged units, wherein:
said selecting is based on the health profile element and on the data structure,
the set of at least two prepackaged units constitute a divided daily dosage for the user, and
the set of at least two prepackaged units includes dietary supplement ingredients designed to alleviate the one or more therapeutic targets of the user; and
generate labels for the set of at least two prepackaged units, wherein the labels are designed to identify correlations between the dietary supplement ingredients included in the set of at least two prepackaged units and the one or more therapeutic targets of the user such that, despite the dietary supplement ingredients included in the set of at least two prepackaged units being usable to alleviate multiple different therapeutic conditions, the labels are designed to emphasize the correlations that are relevant to the one or more therapeutic targets of the user.

18. The computer system of claim 17, wherein each dietary supplement in the data structure is tagged based on a content tagging schema.

19. The computer system of claim 17, wherein the set of at least two prepackaged units of dietary supplement ingredients includes one or more of a female pack, a male pack, an immunity pack, a brain pack, or a sleep pack.

20. The computer system of claim 19, wherein the set of at least two prepackaged units of dietary supplement ingredients includes one or more of an energy pack, an athlete pack, or a digestion pack.

21. (canceled)
